(19) United States
(12) Patent Application Publication
(10) Pub. No.: US 2015/0353821 A1
(43) Pub. Date: Dec. 10, 2015

(54) AN UPCONVERSION FLUORESCENT NANOPARTICLE

(71) Applicant: NATIONAL UNIVERSITY OF SINGAPORE, 21 Lower Kent Ridge Road (SG)

(72) Inventors: Yong ZHANG, Singapore (SG); Qingqing Dou, Singapore (SG); Niagara Muhammad IDRIS, Singapore (SG)

(73) Assignee: NATIONAL UNIVERSITY OF SINGAPORE, SINGAPORE (SG)

(21) Appl. No.: 14/414,008

(22) PCT Filed: Jul. 10, 2013
(86) PCT No.: PCT/SG2013/000290

§ 371 Date: Jan. 9, 2015

Related U.S. Application Data
(60) Provisional application No. 61/670,887, filed on Jul. 12, 2012.

(51) Int. Cl. C09K 11/77; A61K 47/02; G01N 33/58

(52) U.S. Cl. CPC C09K 11/7705; G01N 33/582; A61K 47/02

(57) ABSTRACT

The present disclosure provides upconversion fluorescent nanoparticles comprising: a first nanocrystal layer, a second nanocrystal layer, and an energy absorbing layer disposed between the first nanocrystal layer and the second nanocrystal layer. There is also provided an article of manufacture comprising the upconversion fluorescent nanoparticles, as well as a bio-imaging and/or bio-detection apparatus comprising the upconversion fluorescent nanoparticles. The bio-imaging and/or bio-detection apparatus may further comprise a biomolecule and a source of excitation.

### CROSS-REFERENCE TO RELATED APPLICATIONS

This application is a National Stage of International Application No. PCT/SG2013/000290, filed on Jul. 10, 2013, and published in English as WO 2014/011118 A1 on Jan. 16, 2014. This application claims the benefit and priority of U.S. Provisional Application No. 61/670,887, filed on Jul. 12, 2012. The entire disclosures of the above applications are incorporated herein by reference.

### FIELD OF DISCLOSURE

The present disclosure relates to upconversion fluorescent nanoparticles and an article of manufacture comprising the upconversion fluorescent nanoparticle.

### BACKGROUND

The arduous task of piecing together complex cellular events can now be performed by multiplexing. By throwing a rainbow of fluorescent colours to a cell sample, more than one target can be visualized simultaneously in the same cell, thus allowing several events to be captured in a single snapshot, altogether reducing the amount of reagents, consumables and sample required, besides minimizing sampling errors and eases the inclusion of internal controls. Indeed, such multiplexing capability is made possible with the advent of multicolour fluorescence. Upconversion nanoparticles (UCNs) with highly unusual optical properties and emission wavelength in the ultraviolet (UV), visible (VIS) and near-infrared (NIR) range upon excitation by a single wavelength of NIR light, has come into vogue as a novel group of fluorescent label that is foreseen to overcome current limitations of conventional labels. The use of NIR as an excitation light source gives it a competitive advantage as practically near-zero background visible fluorescence of the sample is generated due to lack of efficient endogenous absorbers in the NIR spectral range besides the fact that most biomolecules do not possess the upconverting property.

When used in a multiplex detection set-up, cross-talking between excitation and emission lights can also be greatly reduced. Moreover, UCNs' capability of multicolour emissions at a single NIR excitation wavelength allows simultaneous excitation of the different colours with ease. In addition, their inherent exceptional photostability feature coupled with low photo-damage (NIR excitation light being generally harmless to biomolecules at low dose) to cells and delicate proteins makes them an attractive tool for long-term live cell imaging. The ability to manipulate the colour output of these UCNs is therefore of particular importance in harnessing their unique optical property to generate novel, superior fluorescent tags for multiplexing applications.

Four colour-UCNs have previously been fabricated by doping Tm, Ho, Er and Yb lanthanide ions into NaYbF₄ and NaYF₄ lattices, albeit at low quality as displayed by their non-uniformity in size, irregularity in shape and having a much lower intensity compared to their bulk counterpart. A method of fine-tuning upconversion emission colour by adjusting the Er/Tm ratio co-doped into the nanocrystals has also been reported. Similarly, a method of synthesizing NaYF₄:Yb,Tm nanocrystals with an overall output colour of blue, purple and red by altering the particle size and their Yb/Tm doping concentrations has also been previously disclosed. Undeniably, however, the above methods are easily succumbed to fluorescence quenching due to cross-relaxation that occurs when Er or Tm ions are doped in the same crystal matrix as other rare earth dopants such that the concentration of Er and Tm ions can only be tuned within a certain range, thus making colour tuning by this method very much limited. As such, it is difficult to obtain multicolour emission UCNs with strong fluorescence by solely adjusting their Er/Tm ratio.

Upconversion emission of NaYF₄ UCNs is size dependent and their green/red emission ratio (f_{g/r}) was affected by coating an undoped α-NaYF₄ shell. Although multicolour UCNs were obtained by manipulating these parameters, nanocrystals emitting different colors were of different sizes, thus hindering their potential for downstream applications.

Multicolour emission upconversion nanospheres based on fluorescence resonance energy transfer (FRET) occurring between UCNs and organic dyes (ODs) or quantum dots (QDs) that have been encapsulated in the silica shell of the UCNs have also been fabricated. However, the multicolour emission was largely dependent on and limited by the FRET efficiency from UCNs to the encapsulated ODs or QDs. Hence, the abovementioned efforts in deriving multicolour UCNs were made at the expense of the particle's upconversion fluorescence intensity.

There is therefore a need for improved upconversion fluorescent nanoparticles.

### SUMMARY

The present disclosure seeks to address at least one of the problems in the prior art, and provides an improved upconversion fluorescent nanoparticle which may be used as an efficient and effective biological label, among other uses.

According to a first aspect, the present disclosure provides an upconversion fluorescent nanoparticle comprising: a first nanocrystal layer, a second nanocrystal layer, and an energy absorbing layer disposed between the first nanocrystal layer and the second nanocrystal layer, wherein each of the first nanocrystal layer and the second nanocrystal layer comprises at least one compound of formula (M₁)ⱼ(M₂)ₖXₙ:(M₃)_{q}, and the energy absorbing layer comprises at least one compound of formula (M₁)ⱼ(M₂)ₖXₙ:(M₃)ᵣ,

wherein
each X is the same or different and is selected from the group consisting of: halogen, O, S, Se, Te, N, P and As;
each M₁, if present is the same or different and is selected from the group consisting of: Li, Na, K, Rb, Cs, Fr, Be, Mg, Ca, Sr, Ba, Ra, O and NH₄;
each M₂ is the same or different and is a metal ion;
each M₃, independently, is the same or different and is selected from the group consisting of Er, Tm, Pr, Ho, Nd, Tb, Eu, Sm, Yb, Ce, Dy, Mo, and Cs;
j is 0≦j≦10; k is 1≦k≦10; n is 1≦n≦10; q is 1≦q≦10; and r is 0≦r≦10.

In particular, j, k, n, q, and r denote the number of M₁, M₂, X, and M₃ elements in one crystal unit cell, respectively. For example, if q or r is 1, only one M₃ element is doped in the layer. When q or r is 2 (or a higher value), two (or more) different M₃ elements are co-doped in the respective layers. Accordingly, j, k, n, q and r do not represent the valency of M₁, M₂, X, and M₃. For example, when the first nanocrystal layer and/or the second nanocrystal layer comprises NaYF₄:Yb,Tm, M₁ is Na, j is 1, M₂ is Y, k is 1, X is F₄, n is 1, M₃ is Yb and Tm co-doped, and q is 2. Likewise, when the energy absorbing layer comprises NaYbF₄:Er, M₁ is Na, j is 1, M₂ is Yb, k is 1, X is F₄, n is 1, M₃ is Er, and r is 1.

M₂ may be any suitable metal ion. For example, M₂ may be transition metal ions, inner transition metal ions, or Group I to VI metal ions. In particular, M₂ may be selected from the group consisting of: Sc, Y, La, Ce, Pr, Nd, Pm, Sm, Eu, Gd, Tb, Dy, Ho, Er, Tm, Yb and Lu.

According to a particular aspect, at least one of the first nanocrystal layer, the second nanocrystal layer, and the energy absorbing layer may comprise at least one emitter ion and at least one absorber ion. In particular, the energy absorbing layer may be saturated with at least one absorber ion.

Each of the first nanocrystal layer and the second nanocrystal layer may comprise any suitable nanocrystal. For example, the first nanocrystal layer and the second nanocrystal layer may comprise any suitable nanocrystal selected from, but not limited to, NaYF₄:(M₃)_{q}, La₂O₃:(M₃)_{q}, La₂O₃:(M₃)_{q}, La₂(MoO₄)₃:(M₃)_{q}, LnF₃:(M₃)_{q}, Y₂O₂S:(M₃)_{q}, Y₂O₃:(M₃)_{q}, TeO₂:(M₃)_{q}, ZrO₂:(M₃)_{q}, LaPO₄:(M₃)_{q}, and LiYF₄:(M₃)_{q}, wherein M₃ and q are as defined above.

The energy absorbing layer may comprise any suitable compound. For example, the energy absorbing layer may comprise at least one of, but not limited to: NaYbF₄:(M₃)ᵣ, La₂O₃:(M₃)ᵣ, La₂O₃:(M₃)ᵣ, La₂(MoO₄)₃:(M₃)ᵣ, LnF₃:(M₃)ᵣ, Y₂O₂S:(M₃)ᵣ, Y₂O₃:(M₃), TeO₂:(M₃)ᵣ, ZrO₂:(M₃)ᵣ, LaPO₄:(M₃)ᵣ, and LiYbF₄:(M₃)ᵣ, wherein M₃ and r are as defined above.

According to a particular aspect, each of the first nanocrystal layer and the second nanocrystal layer may be the same or different and may comprise a nanocrystal selected from the group consisting of: NaYF₄:Yb,Er and NaYF₄:Yb,Tm, and the energy absorbing layer may comprise a compound selected from the group consisting of: NaYbF₄, NaYbF₄:Er, NaYbF₄:Tm and NaYbF₄:Ho.

The upconversion fluorescent nanoparticle may be a NIR-to-visible, NIR-to-NIR, or NIR-to-ultraviolet upconversion fluorescent nanoparticle.

According to another particular aspect, the upconversion fluorescent nanoparticle may comprise at least one biomolecule attached to the nanoparticle. Any suitable biomolecule may be attached to the nanoparticle. For example, the biomolecule may be, but not limited to, protein, nucleic acid, nucleosides, nucleotides, DNA, hormone, amino acid, peptide, peptidomimetic, RNA, lipid, albumin, antibody, phospholipids, glycolipid, sterol, vitamins, neurotransmitter, carbohydrate, sugar, disaccharide, monosaccharide, oligopeptide, polypeptide, oligosaccharide, polysaccharide, and a mixture thereof.

According to a second aspect, the present disclosure provides an article of manufacture comprising an upconversion fluorescent nanoparticle described above. The article of manufacture may be any suitable article of manufacture. For example, the article of manufacture may be, but not limited to, a bio-probe, a carrier for drug delivery, a device for bio-imaging, a bioassay, a device for bio-detection, or an optoelectronic device.

According to a third aspect, the present disclosure provides a bio-imaging and/or a bio-detection apparatus comprising at least one upconversion fluorescent nanoparticle described above, at least one biomolecule, and at least one source of excitation.

The biomolecule may be any suitable biomolecule. For example, the biomolecule may be as described above.

The source of excitation may be any suitable excitation source. For example, the source of excitation may be NIR. In particular, the NIR may be at a wavelength of 980 nm.

The present disclosure also provides a kit comprising at least one upconversion fluorescent nanoparticle described above, or an article of manufacture described above. The kit may, optionally, comprise at least one biomolecule. The biomolecule may be any suitable biomolecule. For example, the biomolecule may be as described above.

### BRIEF DESCRIPTION OF THE DRAWINGS

In order that the disclosure may be fully understood and readily put into practical effect there shall now be described by way of non-limitative example only exemplary embodiments, the description being with reference to the accompanying illustrative drawings. In the drawings:

FIG. 1 shows a schematic design and characterization of sandwich structured UCNs. (a), Top panel: Schematic illustration on the formation of sandwich structured construct to tune UCNs' emission colour with an energy-accumulating B matrix layer sandwiched between two A matrix layers. Sub-structure drawing of the middle B matrix layer is shown on the extreme right. A and B are defined as NaYF₄ and NaYbF₄ matrix, respectively. Bottom panel: Energy transfer diagram of the different lanthanide ions doped in different layers of the ABA construct is shown on the extreme left. Schematic on RGB colour mixing from different shells of the ABA construct to produce multicolour emission UCNs is shown on the right. (b), Representative TEM images of UCNs from a typical synthesis. From left to right: core UCNs, core-shell UCNs and sandwich structured UCNs (scale bar: 50 nm). (c), Particle size distribution statistic histogram of UCNs. From left to right: core UCNs, core-shell UCNs and sandwich structured UCNs. (d), XPS spectra of UCNs. From left to right: core UCNs, core-shell UCNs and sandwich structured UCNs. Inset graphs are the respective elemental scans for each sample. (e), XRD spectra of crystals during formation of sandwich structured UCNs. The layer component of each UCNs are as follows: core UCNs A:Yb,Er, core-shell UCNs A:Yb,Er@B:Er and sandwich structured UCNs A:Yb,Er@B:Er@ A:Yb,Tm. (f), Elemental maps of UCNs at different stages of the sandwich structure formation. From the top to bottom panels, the component of each UCNs are as follows: core UCNs A:Yb,Er, core-shell UCNs A:Yb,Er@B:Er and core-shell-shell UCNs A:Yb,Er@B:Er@A:Yb,Tm;

FIG. 2 shows the emission of uncoated UCNs co-doped with varying amount of Er and Tm emitter ions;

FIG. 3 shows optical images and fluorescent spectra of multicolour emission UCNs. (a) Upconversion spectra of multicolour UCNs. Layer component of UCNs in (a) (from top to bottom) are as follows: A:Yb,Er, A:Yb,Tm, B:Er, A:Yb,Er@B:Tm@A:Yb,Tm, A:Yb,Tm@B:Er@A:Yb,Er, A:Yb,Tm@B:Er@A:Yb,Tm. NaYF₄ is defined as A, and NaYbF₄ is defined as B; (b), Emission of UCNs during shell formation; (c) Emission of UCNs with different shell coatings;

FIG. 4 shows fluorescence emission of sandwich structured UCNs in relation to its core and core-shell UCN counterparts, when its B matrix shell was switched from being the middle to the outermost layer;

FIG. 5 shows emission of UCNs with different thickness of B shell coating;

FIG. 6 shows the roles of different layers in contributing to particle's overall emission. Fluorescence spectra comparison between (a), sandwich structured UCNs having doped versus undoped core; (b), sandwich structured UCNs with different dopant constituents in middle B matrix layer; (c), sandwich structured UCNs with different dopant constituents in outermost A matrix layer;

FIG. 7 shows schematic illustration on simultaneous labelling of multiple sub-cellular targets with multicolour UCNs. Different coloured UCNs, with RGB tunable emission based on its sandwich structured assembly of different combinations of lanthanide doped layers, can be conjugated to antibodies against two cell surface receptors and a microtubular structure. With each colour tagging for a specific cellular target, excitation at a single 980 nm NIR wavelength will yield upconversion fluorescence of a distinct colour for each target, allowing parallel detection of multiple targets with ease;

FIG. 8 shows HER2 single-labelling on live cells with UCNs. Live (a), HER2-overexpressing SK-BR-3 and (b), HER2-low-expressing MCF-7 breast cancer cells were stained for HER2 cell surface receptor with anti-HER2-UCNs-(A:Yb,Tm@B:Er@A:Yb,Tm) (scale bar: 10 μm). (c), A magnified image of SK-BR-3 cell stained with anti HER2-UCNs and (d), its corresponding image with the cell membrane counterstained with Alexa Fluor 488-Concanavalin A conjugate to show subcellular location of the UCN-stained HER2 (scale bar: 5 μm). Red fluorescence indicates upconversion emission from the UCNs under 980 nm excitation, blue fluorescence shows nuclear counterstaining with DAPI while green fluorescence is cell membrane counterstaining with Alexa Fluor 488-Concanavalin A conjugate;

FIG. 9 shows HER2 single-labelling on live cells with UCNs. Live HER2-overexpressing SK-BR-3 and HER2-low-expressing MCF-7 breast cancer cells were stained for HER2 cell surface receptor with anti-HER2-UCNs-(A:Yb,Tm@B:Er@A:Yb,Tm). Red fluorescence (R) indicates upconversion emission from the UCNs under 980 nm excitation while blue fluorescence (B) shows nuclear counterstaining with DAPI. Top panel from each cell line show images taken at low magnification (scale bar: 50 μm) while bottom panel from each cell line show images taken at high magnification (scale bar: 10 μm);

FIG. 10 shows BMPR2 single-labelling on fixed cells with UCNs. Fixed SK-BR-3 cells were stained for BMPR2 cell surface receptor with anti-BMPR2-UCNs-(A:Yb,Tm) (scale bar: 10 μm). Blue fluorescence indicates upconversion emission from the UCNs under 980 nm excitation while nuclear counterstaining with DAPI is pseudocoloured in yellow;

FIG. 11 shows real-time tracking on death of SK-BR-3 cells tagged with anti-HER2-UCNs. Anti-HER2-UCN-stained SK-BR-3 cells was induced to die by withdrawing the usual supply of 5% CO2 and ambient temperature of 37° C. Death process was continuously captured under 980 nm excitation over a 2 hour time period with time-lapse confocal microscopy. Snapshots from the movie at 0 and 2 h of video taking showed changes in the cells' shape, as outlined by their staining with anti-HER2-UCNs. Red fluorescence indicates upconversion emission from the UCNs under 980 nm excitation, blue fluorescence shows nuclear counterstaining with DAPI while green fluorescence is cell membrane counterstaining with Alexa Fluor 488-Concanavalin A conjugate (scale bar: 5 μm);

FIG. 12 shows multiple labelling of sub-cellular targets on live and fixed cells with multicolour UCNs. (a), Fixed 3T3 fibroblast cells were stained separately for cell surface receptors BMPR2 and PDGFR α, and cytoskeletal component microtubule with anti-BMPR2-UCNs-(A:Yb,Tm@B:Er@A:Yb,Tm), anti-PDGFR α-UCNs-(A:Yb,Tm) and anti-α-tubulin-UCNs-(A:Yb,Er@B:Tm@A:Yb,Tm), respectively (scale bar: 10 μm). Red fluorescence in left panel indicates upconversion emission from the anti-BMPR2-UCNs-(A:Yb,Tm@B:Er@A:Yb,Tm), blue fluorescence in middle panel indicates upconversion emission from the anti-PDGFR α-UCNs-(A:Yb,Tm) and red fluorescence in right panel indicates upconversion emission from the anti-α-tubulin-UCNs-(A:Yb,Er@B:Tm@A:Yb,Tm), all under 980 nm excitation. Nuclear counterstaining with DAPI is indicated by the blue fluorescence in left and right panels, while that in middle panel is pseudocoloured yellow. (b), Live 3T3 cells were double-stained for BMPR2 and PDGFR α cell surface receptors with anti-BMPR2-UCNs-(A:Yb,Tm@B:Er@A:Yb,Tm) and anti-PDGFRα-UCNs-(A:Yb,Tm), respectively. Red fluorescence indicates upconversion emission from the anti-BMPR2-UCNs-(A:Yb,Tm@B:Er@A:Yb,Tm) while blue fluorescence indicates upconversion emission from the anti-PDGFR α-UCNs-(A:Yb,Tm), both under 980 nm excitation. (c), Fixed 3T3 cells were triple-stained for BMPR2 and PDGFR α cell surface receptors, and a third microtubular structure with anti-BMPR2-UCNs-(A:Yb,Tm@B:Er@A:Yb,Tm), anti-PDGFR α-UCNs-(A:Yb,Tm) and anti-α-tubulin-UCNs-(A:Yb,Er@B:Tm@A:Yb,Tm), respectively. Red fluorescence indicates upconversion emission from both anti-BMPR2-UCNs-(A:Yb,Tm@B:Er@A:Yb,Tm) and anti-α-tubulin-UCNs-(A:Yb,Er@B:Tm@A:Yb,Tm), green fluorescence indicates upconversion emission from the anti-BMPR2-UCNs-(A:Yb,Tm@B:Er@A:Yb,Tm) while blue fluorescence indicates upconversion emission from the anti-PDGFRα-UCNs-(A:Yb,Tm), all under 980 nm excitation. Nuclear counterstaining with DAPI is pseudocoloured in yellow. Merged images of all the colours show position of the stained cellular structure with respect to each other as well as their distribution within the cell boundary which can be traced from their corresponding bright field images that is shown as insets here in (b) and (c) (scale bar: 10 μm);

FIG. 13 shows single labelling of three sub-cellular targets on fixed cells with different coloured UCNs. Fixed 3T3 fibroblast cells were stained separately for cell surface receptors BMPR2 and PDGFR α and cytoskeletal component microtubule with anti-BMPR2-UCNs-(A:Yb,Tm@B:Er@A:Yb,Tm), anti-PDGFR α-UCNs-(A:Yb,Tm) and anti-α-tubulin-UCNs-(A:Yb,Er@B:Tm@A:Yb,Tm), respectively (scale bar: 10 μm). Red fluorescence in top panel indicates upconversion emission from the anti-BMPR2-UCNs-(A:Yb,Tm@B:Er@A:Yb,Tm), blue fluorescence in middle panel indicates upconversion emission from the anti-PDGFR α-UCNs-(A:Yb,Tm) and red fluorescence in bottom panel indicates upconversion emission from the anti-α-tubulin-UCNs-(A:Yb,Er@B:Tm@A:Yb,Tm), all under 980 nm excitation. Nuclear counterstaining with DAPI is indicated by the blue fluorescence in top and bottom panels, while that in middle panel is pseudocoloured yellow;

FIG. 14 shows double labelling of cell surface receptors on live cells with two-colour multiplexing UCN system Live 3T3 cells were double-stained for BMPR2 and PDGFR α cell surface receptors with anti-BMPR2-UCNs-(A:Yb,Tm@B:Er@A:Yb,Tm) and anti-PDGFR α-UCNs-(A:Yb,Tm), respectively. Red fluorescence indicates upconversion emission from the anti-BMPR2-UCNs-(A:Yb,Tm@B:Er@A:Yb,Tm) while blue fluorescence indicates upconversion emission from the anti-PDGFR α-UCNs-(A:Yb,Tm), both under 980 nm excitation. Nuclear counterstaining with DAPI is pseudocoloured in yellow. Merged images of all the colours show position of the stained cellular structure with respect to each other as well as their distribution within the cell boundary which can be traced from their corresponding bright field images that is shown as an inset here (scale bar: 10 μm);

FIG. 15 shows double labelling of cell surface receptors on fixed cells with two-colour multiplexing UCN system. Fixed SK-BR-3 cells were double-stained for cell surface receptors HER2 and BMPR2 with anti-HER2-UCNs-(A:Yb,Tm@B:Er@A:Yb,Tm) and anti-BMPR2-UCNs-(A:Yb,Tm), respectively. Red fluorescence indicates upconversion emission from the anti-HER2-UCNs-(A:Yb,Tm@B:Er@A:Yb,Tm) while blue fluorescence indicates upconversion emission from the anti-BMPR2-UCNs-(A:Yb,Tm), both under 980 nm excitation. Nuclear counterstaining with DAPI is pseudocoloured in yellow. Merged image of all the colours show position of the stained surface receptors with respect to one another as well as their distribution within the cell boundary which can be traced from their corresponding bright field image that is shown as an inset here (scale bar: 10 μm);

FIG. 16 shows the TEM images of NaYF₄:Er@NaYbF₄ core-shell nanoparticles with different shell thickness. The core-shell molar ratio of each nanoparticle are as follows: (a) 1:0, (b) 1:0.1; (c) 1:0.3, (d) 1:0.5, (e) 1:0.9, (f) 1:1.3, (g) 1:1.7; and (h) 1:2.1;

FIG. 17 shows (a) the fluorescent spectra of NaYF₄:Er@NaYbF₄ core-shell nanoparticles with different shell thickness and (b) changes in the fluorescent intensity of green peak at 542 nm and red peak at 657 nm of the nanoparticles with increasing shell thickness;

FIG. 18 shows the TEM images and size distribution of NaYF₄:Er@NaYbF₄ core-shell nanoparticles with a fixed 1:1.3 core/shell ratio but increasing NaYF₄:Er core size from core-shell 1 to core-shell 4;

FIG. 19 shows (a) the fluorescent spectra of NaYF₄:Er@NaYbF₄ core-shell nanoparticles with a fixed core/shell ratio but increasing NaYF₄:Er core size from core-shell 1 to core-shell 4, and (b) change in the fluorescent intensity of green peak at 542 nm and red peak at 657 nm of the NaYF₄:Er@NaYbF₄ core-shell nanoparticles (at a fixed 1:1.3 core/shell ratio) with increasing core size; and

FIG. 20 shows a modified Bradford assay standard curve for quantitative measurement of HER2 antibodies conjugated to the upconversion fluorescent nanoparticles.

### DETAILED DESCRIPTION

The need for a more efficient biological label to meet their burgeoning utility in rapidly developing multiplexing applications may be realized through the recent advent of upconversion nanoparticles (UCNs). UCNs fabricated to-date, however, are either not displaying strong fluorescence or have limited available colours.

The upconversion fluorescent nanoparticles according to the present disclosure allow for efficient absorption of the excitation energy by the absorber ion-rich energy absorbing layer that then transfers it to the adjacent first nanocrystal layer and second nanocrystal layer on either side of the energy absorbing layer for an improved fluorescence efficiency. By doping different emitters into each of the shells/layers and adjusting their thickness, different colour output tunable based on the RGB colour model may be obtained. Multicolour UCNs with strong emission intensity have been facilely synthesized and used for multiplex detection of three subcellular targets with a single near-infrared excitation wavelength.

The upconversion fluorescent nanoparticle according to the present disclosure may comprise an energy-accumulating matrix sandwiched between two layers of a sandwich structure. For the purpose of the present disclosure, the upconversion fluorescent nanoparticle will be referred to as having a sandwich structure or a core-shell-shell (CSS) structure. The nanoparticles may be highly fluorescent with tunable emission based on the RGB colour model. In particular, the nanoparticles according to the present disclosure provide multicolour emissions of sufficient intensity. Accordingly, the nanoparticles according to the present disclosure may be useful in multiplex detection in view of the multicolour emissions.

According to a first aspect, the present disclosure provides an upconversion fluorescent nanoparticle comprising: a first nanocrystal layer, a second nanocrystal layer, and an energy absorbing layer disposed between the first nanocrystal layer and the second nanocrystal layer, wherein each of the first nanocrystal layer and the second nanocrystal layer comprises at least one compound of formula (M₁)ⱼ(M₂)ₖXₙ:(M₃)_{q}, and the energy absorbing layer comprises at least one compound of formula (M₁)ⱼ(M₂)ₖXₙ:(M₃)ᵣ, wherein

each X is the same or different and is selected from the group consisting of: halogen, O, S, Se, Te, N, P and As;
each M₁, if present is the same or different and is selected from the group consisting of: Li, Na, K, Rb, Cs, Fr, Be, Mg, Ca, Sr, Ba, Ra, O and NH₄;
each M₂ is the same or different and is a metal ion;
each M₃, independently, is the same or different and is selected from the group consisting of Er, Tm, Pr, Ho, Nd, Tb, Eu, Sm, Yb, Ce, Dy, Mo, and Cs;
j is 0≦j≦10; k is 1≦k≦10; n is 1≦n≦10; q is 1≦q≦10; and r is 0≦r≦10.

According to a particular embodiment, the upconversion fluorescent nanoparticle may comprise the first nanocrystal layer, the second nanocrystal layer and the energy absorbing layer in the form of a sandwich structure. In particular, the sandwich structure may comprise a middle NaYbF₄ matrix layer sandwiched between two NaYF₄ matrix layers. The middle energy absorbing layer may achieve the following: (i) its rich content in absorber ions allows for maximum absorption of the excitation energy that is then transferred to the adjacent first nanocrystal layer and second nanocrystal layer lying on either side; (ii) it repairs the surface defects on the nanocrystal core (first nanocrystal layer) and thus minimizes fluorescence quenching; (iii) its own upconversion emission serves as a colour source that can be used to tune the overall output emission colour. In particular, the middle energy absorbing layer may be a NaYbF₄ matrix layer in which the rich content in Yb absorber ions may allow for maximum absorption of the excitation energy that is transferred to the adjacent first nanocrystal layer and second nanocrystal layer. Each of the first nanocrystal layer and the second nanocrystal layer may comprise NaYF₄.

According to a particular embodiment, each of the first nanocrystal layer and the second nanocrystal layer may comprise at least one dopant. The energy absorbing layer may or may not comprise a dopant. The dopant may be an emitter ion and/or an absorber ion. It would be understood by a skilled person that a dopant may be an impurity which is added to a compound in low concentrations to alter some properties of the compound. For example, a dopant may be added in a concentration ranging from one part in a thousand to one part in ten million. It would also be understood that a dopant does not alter the crystal structure of the compound it is added to.

By altering the dopant components in each layer and adjusting the layer thickness, any desired upconversion emission colour can be obtained based on the RGB model. This approach to tune emission colours of the upconversion fluorescent nanoparticles of the present disclosure with strong emission by the sandwich design of an energy-accumulating matrix between layers of a sandwich construct may generate a superior fluorescent tool for a wide range of multiplexing applications. The feasibility for use of the upconversion fluorescent nanoparticles of the present disclosure in multiplex detection was demonstrated by further surface functionalization of these multicolour upconversion fluorescent nanoparticles with different antibodies to target multiple cellular markers simultaneously. The ease of simultaneous excitation of the multicolour upconversion fluorescent nanoparticles with just a single excitation source, as well as other benefits reaped from the upconversion fluorescent nanoparticles' inherent unique optical property, including absence of background fluorescence and use of safe NIR light as an excitation source (thus bypassing the need for potentially cytotoxic UV light normally needed to excite conventional fluorophores such as QDs and green fluorescent proteins), endows these nanoparticles with a significant advantage over other nanomaterials currently used for multiplexing.

The upconversion fluorescent nanoparticle may comprise a first nanocrystal layer and a second nanocrystal layer, wherein the first nanocrystal layer and the second nanocrystal layer may comprise a nanocrystal selected from the group consisting of, but not limited to: NaYF₄:(M₃)_{q}, La₂O₃:(M₃)_{q}, La₂O₃:(M₃)_{q}, La₂(MoO₄)₃:(M₃)_{q}, LnF₃:(M₃)_{q}, Y₂O₂S:(M₃)_{q}, Y₂O₃:(M₃)_{q}, TeO₂:(M₃)_{q}, ZrO₂:(M₃)_{q}, LaPO₄:(M₃)_{q}, and LiYF₄:(M₃)_{q}, wherein M₃ and q are as defined above.

The energy absorbing layer may be any suitable layer. For example, the energy absorbing layer may comprise at least one of, but not limited to: NaYbF₄:(M₃)ᵣ, La₂O₃:(M₃)ᵣ, La₂O₃:(M₃)ᵣ, La₂(MoO₄)₃:(M₃)ᵣ, LnF₃:(M₃)ᵣ, Y₂O₂S:(M₃)ᵣ, Y₂O₃:(M₃)ᵣ, TeO₂:(M₃)ᵣ, ZrO₂:(M₃)ᵣ, LaPO₄:(M₃)ᵣ, and LiYbF₄:(M₃)ᵣ, wherein M₃ and r are as defined above.

According to a particular embodiment, the energy absorbing layer may not comprise a dopant, in that r is 0. For example, when the energy absorbing layer is not required to contribute to the emission colour, the energy absorbing layer need not be doped with emitter ions. However, the energy absorbing layer may comprise an absorber either as a dopant or by selecting an appropriate M₂.

According to a particular aspect, each of the first nanocrystal layer and the second nanocrystal layer may be the same or different and may comprise: NaYF₄:Yb,Er or NaYF₄:Yb,Tm, and the energy absorbing layer may be selected from the group consisting of: NaYbF₄, NaYbF₄:Er, NaYbF₄:Tm and NaYbF₄:Ho.

### Sandwich Structured UCN Synthesis

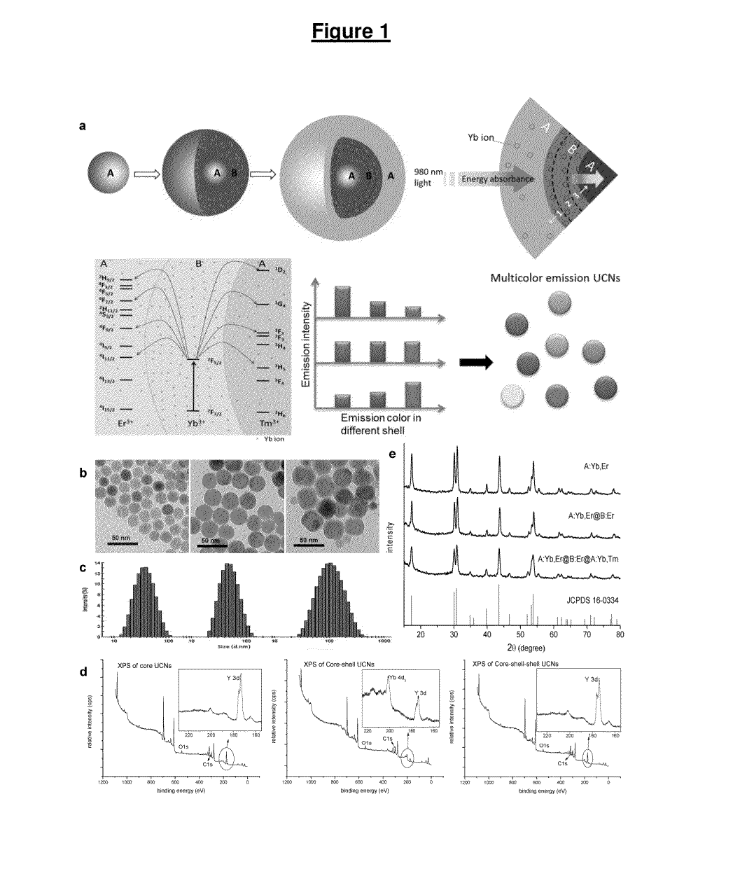

The first nanocrystal layer and the second nanocrystal layer will be denoted by “A” while the energy absorbing layer will be denoted by “B”. As schematized in FIG. 1a, synthesis of the sandwich structured nanoparticles of the present disclosure was performed in a stepwise process, beginning with formation of the A matrix (for example, NaYF₄ doped with emitters) core, followed by its coating with a layer of B matrix (for example, NaYbF₄ doped with emitters) shell, and finally coating of another A matrix shell onto the B matrix to form an ABA sandwich structured UCN.

According to a particular embodiment, the emitter dopant concentration was fixed to 2 mol % Er and 0.3 mol % Tm in both the A and B matrices, while that of Yb sensitizer was set to 20 mol % and 100 mol % (as emitter) in the A and B matrix, respectively. By this method, uniform-sized spherical nanoparticles were obtained as evident from their transmission electron microscopy (TEM) images (FIG. 1b). Here, a gradual increase in the particles' size from about 15 to 43 nm was observed as they underwent a structural transition from core to sandwich structured UCNs, thus suggesting successful formation of the sandwich structure. With such a size increment, it can thus be inferred that the coating seemed to produce an average shell thickness of around 5 nm. Admittedly, actual visualization of these shells on the core-shell or sandwich structured particles was not evident from the TEM images. This may be attributed to the fact that there was no appreciable Z-contrast between the Y and Yb element dominating the A and B matrix, respectively, as their ionic size are close enough to allow them occupy the same lattice site in the crystal. Hence, formation of shells on the UCN core, as reflected by changes in their size, was subsequently further confirmed by particle size measurement with a zeta-nanosizer that revealed an increase in the average diameter of the nanoparticles over time during the shell coating process (FIG. 1c). Additionally, the result also suggests that the particles were monodispersed after formation of their first and second shells, thus furnishing further experimental evidence on the successful formation of the sandwich structure. It was also noted that there is likelihood that the reactants newly added during the shell coating process would form new small particles by nucleation rather than get deposited on the surface of pre-existing core UCNs. However, since this largely depends on the energy equilibrium of the reaction, the shell formation is anticipated to be the dominant product here as the energy barrier that needs to be overcome in forming a new crystal is much higher compared to that required for ions deposition on an existing core.

The composition and nanostructure of the formed UCNs were further examined by X-ray photoelectron spectroscopy (XPS) and X-ray diffraction (XRD). As the particles' average shell thickness is around 5 nm, Y and Yb ions in each sample can be conveniently traced by XPS since XPS is sensitive to the top 5 nm of a sample. Here, XPS wide scan was recorded for element correction based on the characteristic peaks of carbon and oxygen. The elemental scan was focused on the Y and Yb elements since they are the characteristically dominant elements found at different stages of the sandwich structure formation. As shown in the middle panel of FIG. 1d, it is obvious that the peak around 200 eV that corresponds to 4d₃ electrons of Yb in the middle NaYbF₄ matrix layer is much higher than that observed in the core UCNs (FIG. 1d, left panel) and sandwich structured UCNs (FIG. 1d, right panel). On the other hand, peaks around 175 eV that represent Y 3b electrons, are relatively higher in the core UCNs and sandwich structured UCNs as compared to their core-shell UCN counterpart. These XPS results not only vindicate that the shells were successively coated on the core, but that they do not diffuse away, thus suggesting for their firm deposition on the core surface after coating. Additionally, the fact that the Y ions were rich only after coating the layer of B shell clears the doubts on ions immigration within the nanoparticles that might have occurred when they are placed in hot solutions, altogether negating the assumption that diffusion and segregation of different ions could have occurred. Indeed, this was further confirmed by comparing the fluorescence spectra of the sandwich structured UCNs of the present disclosure to that of core UCNs commonly synthesized previously. For this, tri-doped UCNs (A:Yb,Er,Tm with elemental ratio Y:Yb:Er:Tm=53.3:47.3:1.3:0.1) and sandwich structured UCNs (A:0.2 Yb, 0.02 Er@B:0.02 Er@A: 0.2 Yb, 0.003 Tm) having the same amount of ion content were compared. It was found that the emission of tri-doped UCNs was very dim as opposed to the sandwich structured ones, and that the recorded fluorescence intensity was even much lower than UCNs co-doped with Yb and Er/Tm, due to cross-relaxation that occur between dopants when they are present at high ion concentration. Thus, this corroborates previous notion that there was no ions immigration during the shell coating reaction.

Further confirmation of the formation of the sandwich structure (core-shell-shell structure) was done by performing elemental mapping of the absorber element on the nanoparticle surface by Time-of-Flight Ion Mass Spectrometry (TOF-SIMS). At different stages of the shell formation, the concentration of the absorber element is expected to be much different and this can be observed based on the brightness of the elemental maps which increased proportionally to the concentration of the elements present. According to a particular embodiment, the absorber element may be selected to be Yb. The elemental maps may measure 300×300 μm² areas. The results of the TOF-SIMS for investigating the concentration of Yb on the upconversion fluorescent nanoparticle having a core-shell-shell structure is as shown in FIG. 1f. In particular, the concentration of the Yb isotopes on the surface of the core-shell nanoparticles was observed to be higher than that of the core or the upconversion fluorescent nanoparticles having the sandwich structure, which indicate that the Yb element was found only to be rich in the middle energy-absorbing layer, i.e. the NaYbF₄ layer, thus proving the formation of a core-shell-shell structure.

Additional characterization of the particles based on XRD patterns suggest that single-crystalline hexagonal phase nanocrystals were produced at every stage of the reaction (FIG. 1e). The right shifted XRD peaks observed here indicate that the average size of the crystal unit cell shrank slightly after treatment of the sandwich structure with Yb substituted matrix, justified by the fact that Yb is smaller in ionic size (i.e. 1.125 Å) than the previous occupant Y (1.159 Å).

### Sandwich Structure Strategy for Colour Tuning

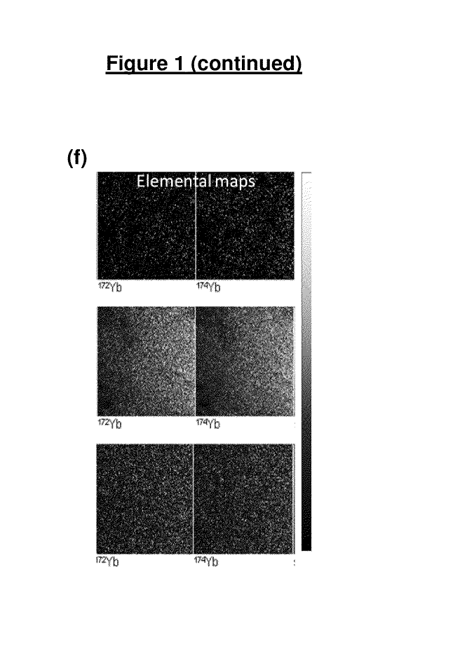

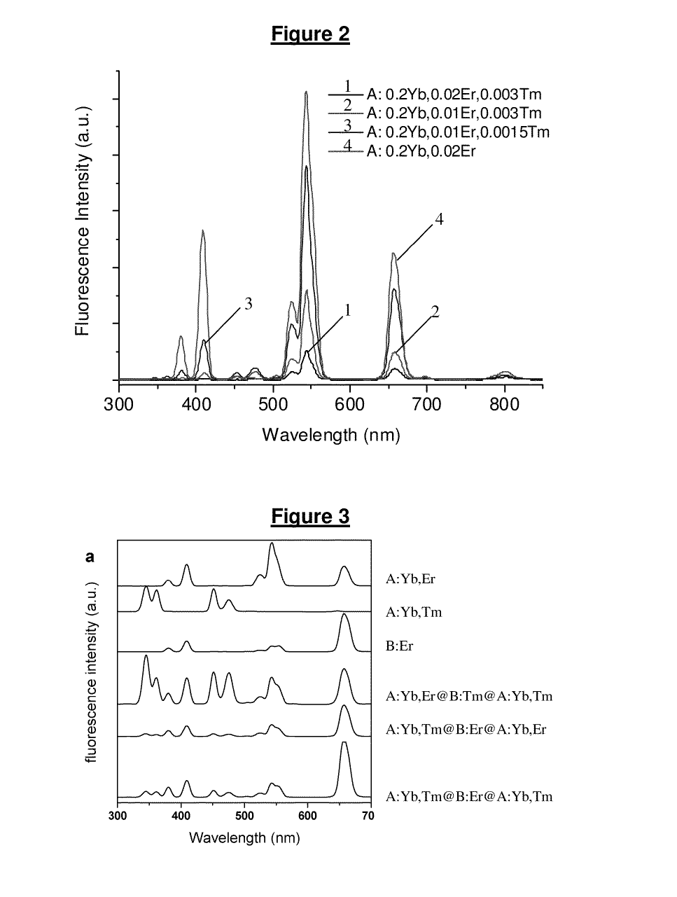

Tuning the colour output of UCNs cannot be done simply by superposing the emission peaks of the dopants, such as of Er and Tm (co-doped in a nanoparticle) over each other as each peak on the spectrum corresponds to an energy level. For instance, the emission peaks at 450 and 475 nm were assigned to the ¹D₂→³F₄ and ¹G₄→³H₆ transitions of Tm ions while emission peaks at 409, 520, 541, and 653 nm were assigned to the transitions of Er ions from ⁴H_{9/2}→⁴I_{15/2}, ⁴H_{11/2}→⁴I_{15/2}, ⁴S_{3/2}→⁴I_{15/2} and ⁴F_{9/2}→⁴I_{15/2}. Hence, doping two different emitter ions into one nanoparticle may not necessarily produce an emission spectrum whose intensity is simply the sum of their individual emitter's fluorescence. On the contrary, a drop in the absolute emission intensity of UCNs co-doped with both Er and Tm emitter ions was observed when compared to commonly synthesized UCNs (A:0.2Yb,0.02Er) having only a single species of emitter doped (i.e. Er) (FIG. 2). To shed more light on this, different Er-to-Tm ratio doped UCNs were synthesized, while keeping the absorber ion Yb concentration constant at 20 mol % ratio for all UCN samples (so as to ensure that the same amount of excitation energy absorbed by the UCN is available to the emitters). First, UCNs that were co-doped with Er and Tm emitter ions at a concentration that was found optimal when these ions were individually doped into the nanocrystals (i.e. 0.02Er, 0.003Tm), were prepared. Subsequently, other UCNs co-doped with half the amount of Er ions but optimal Tm amount (0.01 Er, 0.003Tm), or those having both Er and Tm ions halved (0.01Er, 0.0015Tm), were also synthesized. As shown in the recorded spectra in FIG. 2, UCNs having a higher net amount of Er and Tm emitter ions display a lower fluorescence intensity than those having a lesser net amount of these emitter ions, with those having the least amount of net emitter ions of 0.01 Er, 0.0015Tm emitting the highest fluorescence amongst the Er-Tm co-doped samples (though not reaching the same intensity level as those UCNs doped with only a single emitter ion Er). This is attributed to the loss in energy transferred from the radiation source due to cross-relaxation between ions, thus leading to less number of photons whose energy level is high enough to generate fluorescence as it falls down to ground state. Therefore, these observations indicate that tuning UCNs' emission by Er/Tm co-doping is an inefficient way in producing multicolour UCNs of strong intensity. Indeed, the tri-doped system requires striking a balance between doping enough Yb absorber ions so as to absorb as much of the radiation energy for conversion to an excited photon energy, while maintaining it below the level where the saturation effect of Yb ions sets in. With the upconversion fluorescent nanoparticle of the present disclosure, this can be bypassed by having a middle energy absorbing layer that can be assigned to have more absorber ions such as Yb ions packed in for maximum energy absorption, and adjacent first nanocrystal and second nanocrystal layers doped with different emitter ions in which the absorbed energy can be efficiently transferred to, for an unsuppressed emission.

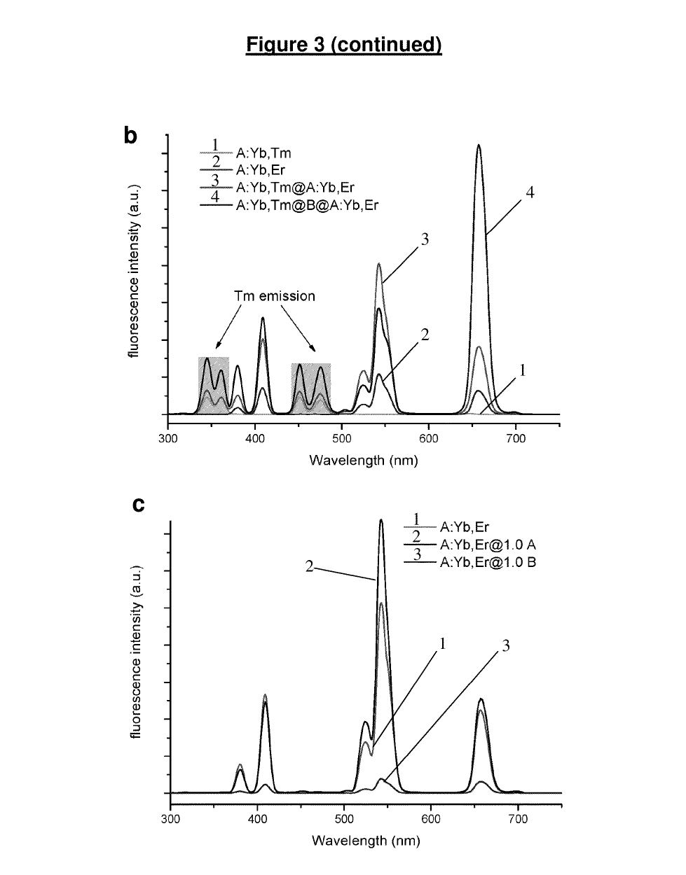

The feasibility of colour tuning with an energy-accumulating matrix sandwiched between two layers of a sandwich structure was explored. In this sandwich design, a NaYbF₄ matrix is assigned to the middle energy absorbing layer for maximum energy absorption, while adjacent first nanocrystal and second nanocrystal layers are doped with emitter ions in which the absorbed energy can be efficiently transferred to. Sandwich structured nanoparticle samples having different combination of emitters doped into the different layers were synthesized. In all these samples, the thickness of each layer and thus the resultant size of the sandwich structured UCNs were made comparable by keeping the total amount of chemicals used to fabricate each layer constant under the same reaction condition. By changing the dopants in each shell, tuning of the emission colours was achieved, as shown in FIG. 3a. It was interesting to note here that the Tm emission was greatly increased when doped into the sandwich structure (FIG. 3a; A:Yb,Er@B:Tm@A:Yb,Tm). This is rather surprising given the fact that the optimal Tm concentration range for blue upconversion fluorescence is very low (around 0.3% as compared to Er at 2%), that it is usually difficult to tune the emission intensity of Tm doped in conventional core-shell structure. In the sandwich structure of the upconversion fluorescent nanoparticles though, increase in Tm emission intensity was easily achieved without having to dope much more of the Tm ions. Indeed, this was further confirmed by another experiment whereby UCNs having sandwich structure demonstrated much higher absolute fluorescence intensity than its core-shell UCN counterpart or the core itself (FIG. 3b), thus indicating yet another benefit that sandwich structure has on UCNs. This enhancement can be attributed to two reasons: (i) a chain repair of surface defects on the core (first nanocrystal layer) by the first shell (energy-absorbing layer) and on the first shell by the second shell (second nanocrystal layer); (ii) sandwich structure provides a means to dope a high amount of absorber ions into the UCNs without the concern that its saturation effect may have on fluorescence quenching by having a middle energy absorbing layer that can be assigned to have more absorber ions packed in for maximum energy absorption, and adjacent layers doped with emitter ions in which the absorbed energy can be efficiently transferred to, for an unsuppressed emission. Therefore, with different emitters doped in each layer, upconversion emission of the upconversion fluorescent nanoparticle of the present disclosure can be tuned based on the RGB colour model without suppression arising from cross-relaxation between the lanthanide dopants.

### Striking a Balance Between the Shielding and Enhancing Effect of a Shell Coating

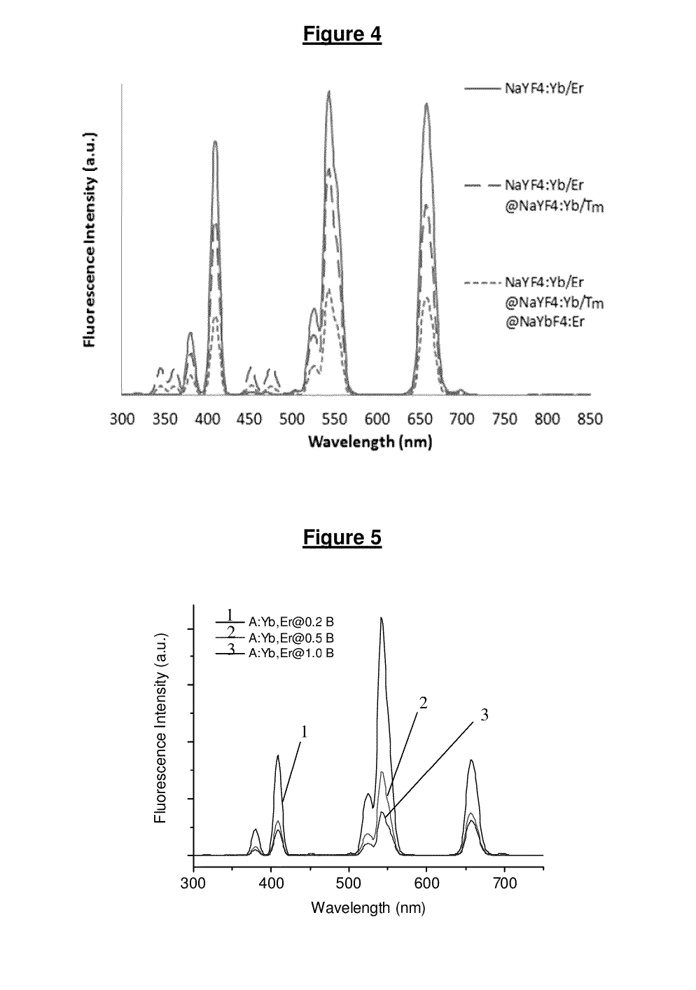

In the sandwich structural design, the middle energy absorbing layer of B matrix shell plays two different and contrasting roles in both shielding and enhancing the fluorescence output of the UCN. According to these roles, it can thus be further sub-divided into three layers, as drawn by the dashed lines in the schematic of FIG. 1a. The outer and innermost sub-layers are believed to have an enhancing effect on the nanoparticle's overall emission, while the middle sub-layer plays the contrary role of having a shielding effect on the fluorescence emanating from the core. The B matrix shell was switched from being the middle layer to be the outermost second nanocrystal layer such that the newly sandwich structured UCNs now has layer components of A:Yb,Er@A:Yb,Tm@B:Er. Indeed, such a switch resulted in a decrease in the intensity of all the emission peaks of the resultant sandwich structured A:Yb,Er@A:Yb,Tm@B:Er nanocrystals as compared to its nanoparticle core or the core-shell nanoparticle (FIG. 4). This highlights the importance of the B matrix position within the multilayered nanoparticle as it plays a strong influence on the emission fluorescence of the resultant nanoparticle. To ascertain that such a decrease in fluorescence intensity is indeed due to the layer of B matrix shell being placed at the outermost position, A:Yb,Er core nanocrystals were coated with just a layer of B matrix. Comparison of its emission fluorescence before and after the coating process revealed a drop in all the emission peaks after the layer of B matrix was coated onto the core (FIG. 3c). This was similarly observed in other series of experiments whereby core-shell nanostructures having the same A matrix core but coated with a layer of either A matrix shell (having 20 mol % Yb) or B matrix shell (having 100 mol % Yb, as emitter)) of varying thickness were synthesized and compared. It was found that the fluorescent intensity of those coated with the A matrix shell was almost 15 times stronger than that coated with the B matrix shell of the same thickness (FIG. 3c), despite having a similar average size, as measured by DLS. When the shell-to-core total ion mole ratio was decreased to 0.5 and then further to 0.2, the fluorescent intensity increased correspondingly (FIG. 5), though never reaching the same intensity level as the core. Taken together, these results suggest that the layer of absorber-rich (Yb-rich) B matrix shields fluorescence that is emanating from the inner layers when it is placed at the outermost layer. This shielding is mainly due to the saturation effect of Yb absorbers. When placed at the outermost position, the strong absorption of incident NIR light by the abundant Yb absorber ions packed in the B matrix was not efficiently transferred to the Er or Tm emitter ions doped in the inner layers of the nanocrystals. Indeed, the efficiency in which energy is converted from the radiation source to the UCNs depends largely on its concentration of the Yb absorber ions. This conversion efficiency increases with increasing Yb concentration till it reaches a saturation point, beyond which its overly dominant presence leads to a shorter distance between Yb and Er/Tm emitter ions, such that energy back transfer from Er to Yb ions will now occur. This greatly reduced the amount of NIR excitation energy that is capable of reaching the inner layers, thereby accounting for the decrease in fluorescence emanating from these inner layers. Although upconversion emitter ions such as Er or Tm can be directly excited by NIR light itself, their absorption cross-section in the NIR region is ten times lower than that of Yb ions. Such low absorption cross-section of Er and Tm emitter ions in the NIR region, coupled to poor energy transfer from Yb ions to these emitter ions, results in negligible contribution of the shielded inner core towards the overall fluorescence intensity of the nanocrystals. Thus, even though coating a layer of B matrix shell on the UCN core is expected to minimize fluorescence quenching by repairing surface defects and isolating it from surrounding solvent molecules and surface ligands, it did not contribute to a fluorescence enhancement here as evident from the suppressed emission of the core-shell UCNs. On the other hand, coating a layer of A matrix shell enhanced fluorescence of the UCN inner core as the amount of Yb ions doped is optimal here.

### Role Played by Each Layer in Contributing to Nanoparticle's Overall Emission

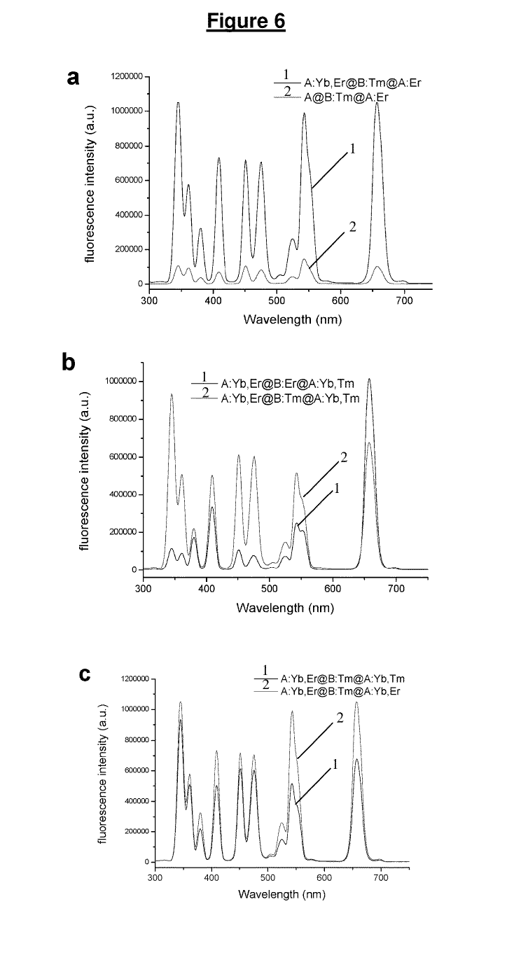

The role played by each layer in contributing to the particle's overall emission was also observed. The role of the first nanocrystal layer (core) was first examined by comparing sandwich structured UCNs having the same shells but different core components, in which one was doped while the other was left undoped as pure A matrix core. Although the two types of particles revealed a similar emission profile, those particles having an undoped core displayed fluorescence that was ten times weaker than its doped counterpart (FIG. 6a). The fluorescence emitted from the doped core does not seem to be suppressed even when two layer of shells are coated over it, one of which is the middle energy absorbing B matrix layer which may be rich in absorber ions such as Yb ions. Thus, this clears lingering doubt on whether high amount of absorber ions such as Yb ions packed into the middle layer would lead to it absorbing fluorescence emitted from the adjacent first nanocrystal and second nanocrystal layers. Backed by the high absorption cross-section of Yb ions in the NIR region, the obvious increase in the fluorescence intensity of the UCNs before and after growth of an A layer matrix over the B layer matrix may indicate that the middle B layer serves as a sink for accumulating the absorbed excitation energy that will then be transferred to adjacent layers on the inner and outer sides simultaneously.

The role of dopants in the middle energy absorbing layer was probed to assess its contribution to the particle's overall emission intensity and profile. Here, two types of sandwich structured UCNs having the same core (A:Yb,Er) and outermost layer (A:Yb,Em) but a differently doped middle layer was examined. As shown in FIG. 6b, the dopant constituents in the middle layer of B matrix contributed more than one fold of its typical emissions to the overall emission intensity, thus indicating the importance of the middle layer in contributing to the particle's emission, albeit at a smaller extent than that previously observed from the core. Lastly, the role played by the outermost layer in contributing to the particle's overall emission was investigated by comparing sandwich structured UCNs that have the same core (A:Yb,Er) and middle layer (B:Tm) components but a different outermost layer. It is clearly shown here that the typical Er emission peaks around 550 and 650 nm were slightly enhanced in particles having the A:Yb,Er components as its outermost layer as compared to those having the A:Yb,Tm components (FIG. 6c). Hence, by studying the emission behaviour of each layer separately, their individual role in contributing to the particle's emission spectra was revealed, and the outermost layer seemed to show the weakest influence here, probably due to the fact that they are thinner compared to the other two core and middle layers.

Energy Transfer from Energy Absorbing Layer to First Nanocrystal Layer and/or Second Nanocrystal Layer

In the upconversion fluorescent nanoparticles of the present disclosure, the energy absorbed in the energy absorbing layer is transferred to the first nanocrystal layer and/or to the second nanocrystal layer. This was confirmed by investigating the energy transfer from the energy-absorbing layer (shell) to the first nanocrystal layer (core). Energy transfer between absorber and emitter ions doped within the same layer was excluded by having a core-shell nanoparticle with the absorber and emitter ions doped in separate layers. Core-shell upconversion nanoparticles of NaYF₄:Er@NaYbF₄ were synthesized, in which only the energy transfer from shell (doped with Yb absorber) to core (doped with Er emitter) is allowed. By altering the shell thickness and core size of the core-shell nanoparticles, optimal thickness of the NaYbF₄ layer and size of the core were obtained to achieve the highest fluorescence. In a typical synthesis procedure, NaYF₄:Er core was synthesized following any suitable protocol such as that described below (“A (NaYF₄) core UCN synthesis”), and the dopant concentration was fixed to 2 mol % Er. The method used to coat the NaYbF₄ layer on the NaYF₄:Er core is also similar to that of the core-shell UCN synthesis protocol described below (“AB (NaYF₄@NaYbF₄) core-shell UCN synthesis”), except that the amount of shell precursor was adjusted according to the core/shell ratio in each sample.

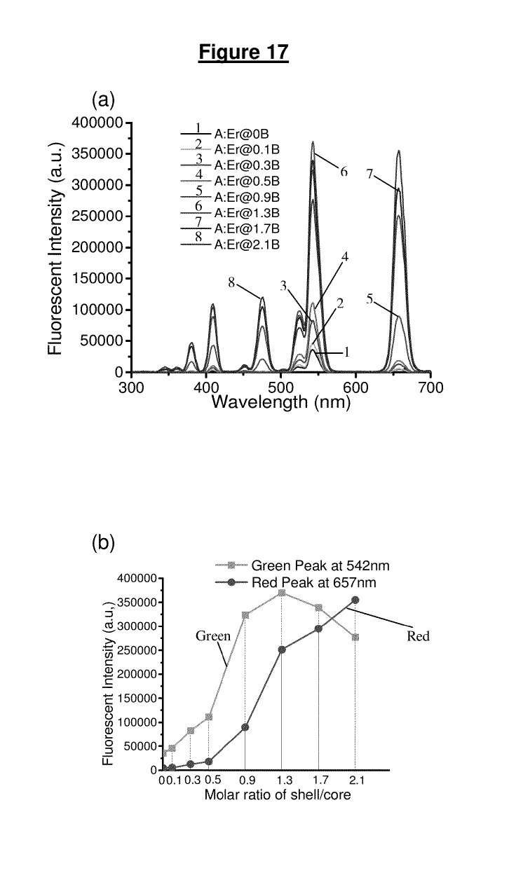

A set of NaYF₄:Er@NaYbF₄ core-shell UCNs with different thickness of NaYbF₄ shell was first synthesized to investigate the factor on shell thickness in energy transfer from NaYbF₄ layer to the core NaYF₄:Er. From the TEM images as shown in FIG. 16, it is observed that there is a gradual increment in the size of the core-shell UCNs with increasing amount of precursors used in the NaYbF₄ shell synthesis, thus indicating the successful coating of the NaYbF₄ layer. Energy-dispersive X-ray spectroscopy (EDX) analysis of Y to Yb ratio also matches well with the nominal ratio of chemicals used in the shell synthesis (see Table 1 below).

TABLE 1
Nominal and EDX measured Y/Yb ratio

| | | | | | |
|---|---|---|---|---|---|
| Nominal Y/Yb | 1:0 | 1:0.1 | 1:0.3 | 1:0.5 | 1:0.9 |
| Y/Yb in EDX | 1:0 | 1:0.127 | 1:0.337 | 1:0.646 | 1:1.086 |

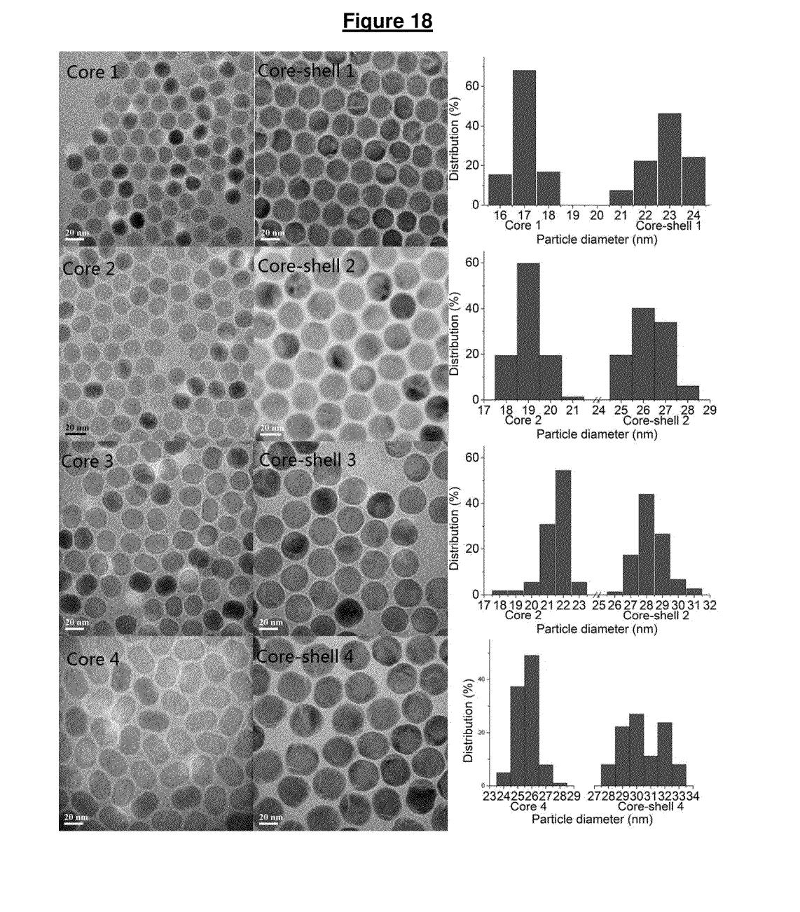

Fluorescent spectra of the particles showed that the green emission peak at 542 nm, which corresponds to ⁴S_{3/2} to ⁴I_{15/2} transitions of Er ions, firstly increased with increasing thickness of the NaYbF₄ shell before reaching its peak intensity at a core/shell ratio of 1:1.3, and then beyond which it decreased with further increment in the NaYbF₄ shell thickness (FIG. 17). This indicates that the NaYbF₄ shell plays two different and contrasting roles in both enhancing and shielding the fluorescence of the core-shell nanoparticles. The NaYbF₄ shell enhances the fluorescence by absorbing energy and then transferring it to Er ions doped in the core. When the shell thickness increases, more energy is absorbed by the Yb ions and consequently more of this energy gets transferred to Er ions, resulting in an increment in the particle's fluorescent intensity. However, when the NaYbF₄ shell reaches a thickness greater than a critical value, it now exerts the contrasting role of shielding the fluorescence emitted from the core, thus explaining for the decrease in the 542 nm peak of Er being observed in FIG. 17. Considering these two contrasting effects, the optimal core/shell ratio for energy transfer from NaYbF₄ layer to core is 1:1.3. In contrast, the cooperative upconversion emission peak at 657 nm of Yb increased with increasing thickness of the NaYbF₄ shell, since the cooperative upconversion is directly proportional to the Yb concentration.

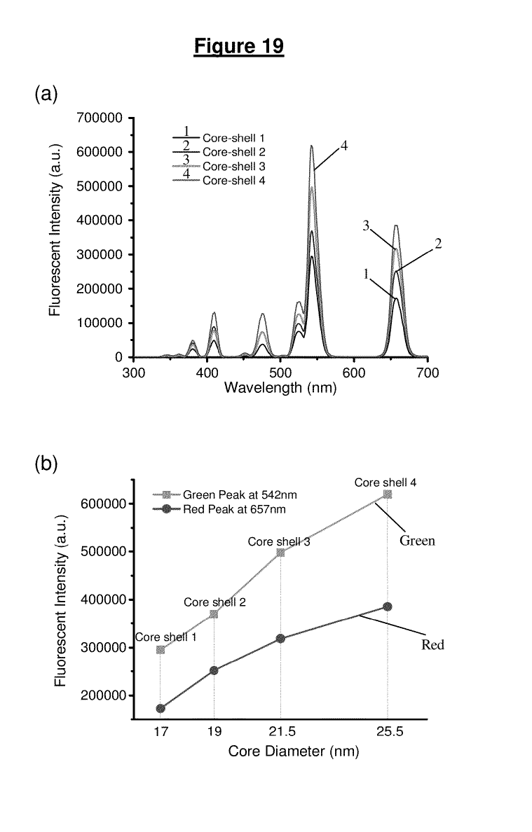

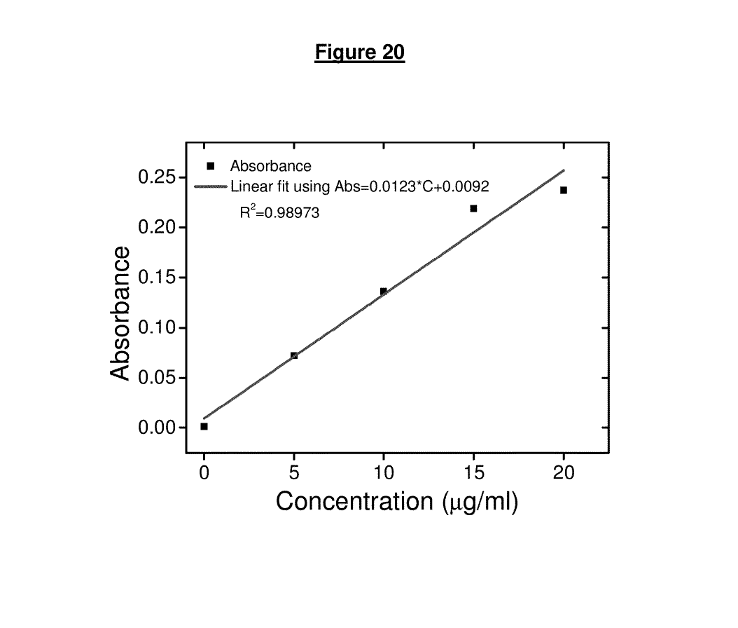

In the second approach, the factor on core size was investigated to assess its effect on the energy transfer from NaYbF₄ layer to core. For this, a set of NaYF₄:Er@NaYbF₄ core-shell UCNs with different core sizes were synthesized (FIG. 18). Based on the previous study on shell thickness, the core/shell ratio is fixed to the optimal ratio of 1:1.3. The increase in size from core to core-shell with narrow distribution indicates the successful coating of the NaYbF₄ layers. The fluorescence intensity of the core-shell UCNs increased with an increase in the core size (FIG. 19). This may be attributed to the fact that with an increase in the nanoparticle size, the crystal defects found on the surface of these nanoparticles also decreases, thus reducing the quenching effect usually known to be caused by crystal defects, thereby resulting in a fluorescence enhancement.

Multiplex Detection of Cellular Markers with Multicolour Antibody-Conjugated UCNs

According to a particular aspect, the upconversion fluorescent nanoparticle may further comprise at least one biomolecule attached to the nanoparticle. For example, the biomolecule may be selected from, but not limited to, protein, nucleic acid, nucleosides, nucleotides, DNA, hormone, amino acid, peptide, peptidomimetic, RNA, lipid, albumin, antibody, phospholipids, glycolipid, sterol, vitamins, neurotransmitter, carbohydrate, sugar, disaccharide, monosaccharide, oligopeptide, polypeptide, oligosaccharide, polysaccharide, or a mixture thereof.

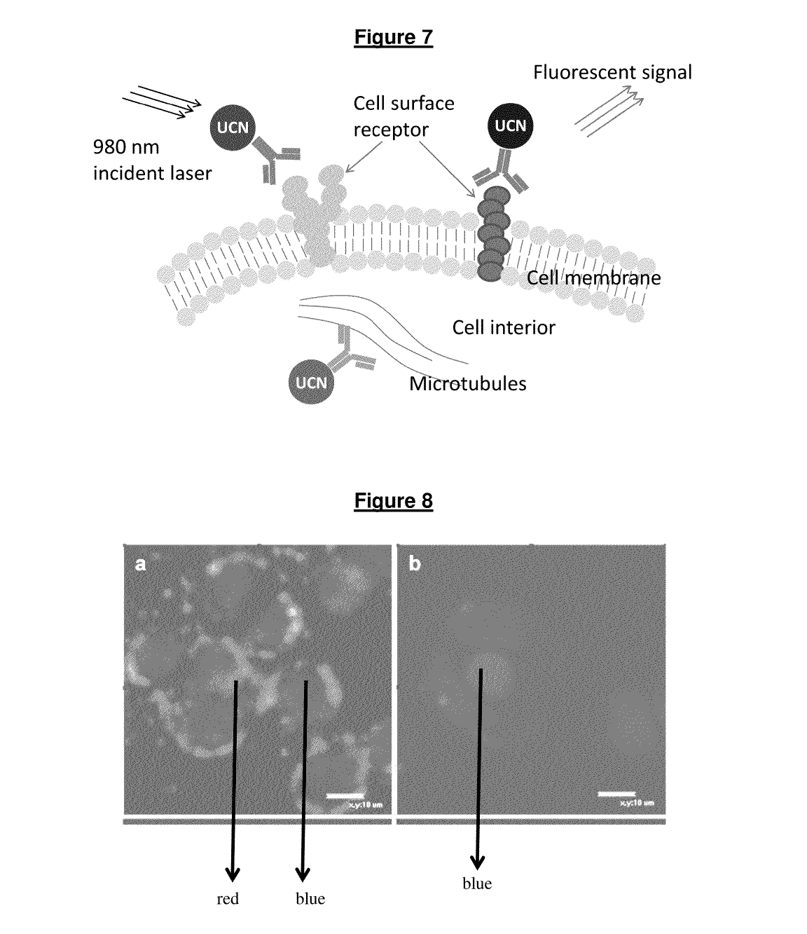

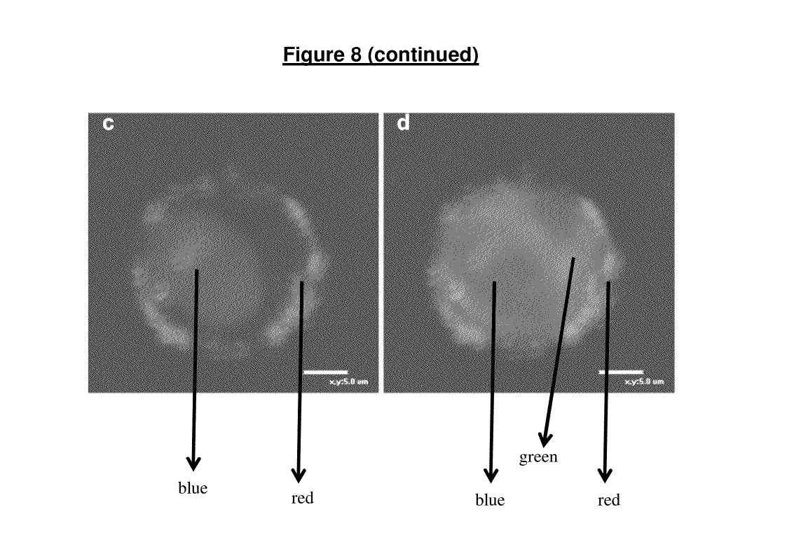

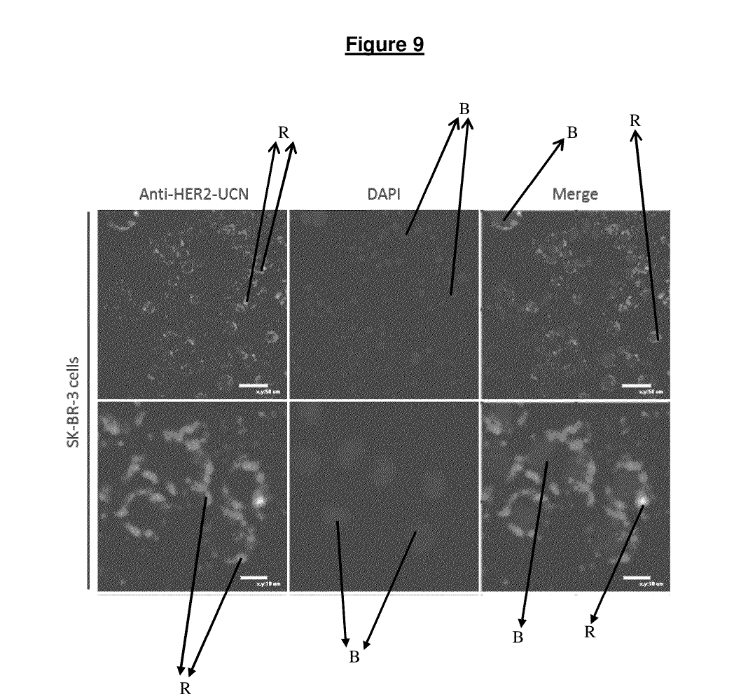

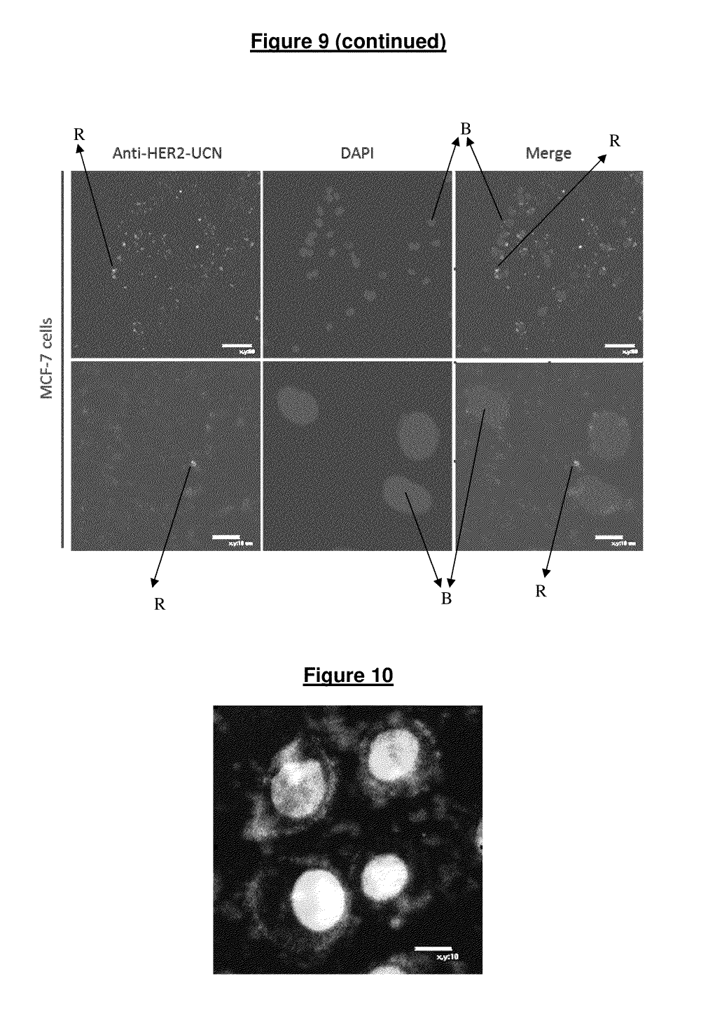

Using the same sandwich base construct as described above, different colours that are tunable based on the RGB colour model with different combination of emitters doped into the different layers can thus be assigned to these UCNs, essentially producing particles of multicolours. According to a particular embodiment, the multicolour UCNs' feasibility as a promising candidate for multiplex detection of cellular markers (as schematized in FIG. 7) was assessed in a stepwise fashion, beginning first with detection of a single target—the human epidermal growth factor receptor 2 (HER2)—a cancer marker that is overexpressed on the surface of some breast cancer cells such as SK-BR-3. HER2 antibody covalently conjugated to carboxyl group functionalized UCNs-(A:Yb,Tm@B:Er@A:Yb,Tm) were incubated with live SK-BR-3 cells for 4 hours. Excitation of these cells with a 980 nm NIR light revealed a strong predominantly red upconversion fluorescence that appeared as a bright rim of staining around the cells' nuclear region (traced based on double stranded DNA staining with 4′,6-diamidino-2-phenylindole (DAPI)) (FIG. 8a, c and FIG. 9). Using Alexa Fluor 488-Concanavalin A conjugate to stain the cell membrane, we were able to confirm that these anti-HER2-UCNs were localized to the outer face of the plasma membrane (FIG. 8d), vindicating the specificity of the UCN-antibody conjugate for its cell-surface receptor target. In stark contrast, HER2-low-expressing MCF-7 breast cancer cells exposed to UCNs under the same staining conditions produced minimal of such fluorescence upon 980 nm laser excitation (FIG. 8b and FIG. 9). This suggests that the UCN-antibody conjugates bind to its target in a specific manner and are useful in detecting differentially or altered expressed level of a cellular marker of interest between different samples, as exemplified here by HER2 labelling on different cell lines. Robustness of the UCN technology as a labelling tool to detect diverse array of cellular markers was also evaluated by conjugating UCN of another colour (A:Yb,Tm—showing predominantly blue emission) to a second cell surface receptor antibody—anti-bone morphogenetic protein receptor type II (BMPR2). Incubating SK-BR-3 cells with these UCNs showed effective staining of BMPR2 on the surface of the cells (FIG. 10).

Besides specificity, another important feature that is critical for most fluorescent-based applications is photostability. This is especially so in live cell imaging that real-time monitors the dynamics of cellular events, whereby use of antifade mounting medium normally employed to provide protection against photobleaching now becomes impractical. Herein, the above study was extended by using anti-HER2-UCNs as a labelling agent to monitor death of SK-BR-3 cells when their culture condition was disrupted upon withdrawing the usual supply of 5 carbon dioxide (CO₂) and ambient temperature of 37° C. Death process of these anti-HER2-UCN-stained SK-BR-3 cells, presumably by necrosis, was tracked using time-lapse confocal microscopy. Swelling and eventual plasma membrane rupturing of the dying cells was unobtrusively captured in real-time over a 2 h time course (FIG. 11), during which the UCNs' fluorescent signal was very stable despite being within the culture medium milieu that is known to affect photostability of conventional fluorophores.

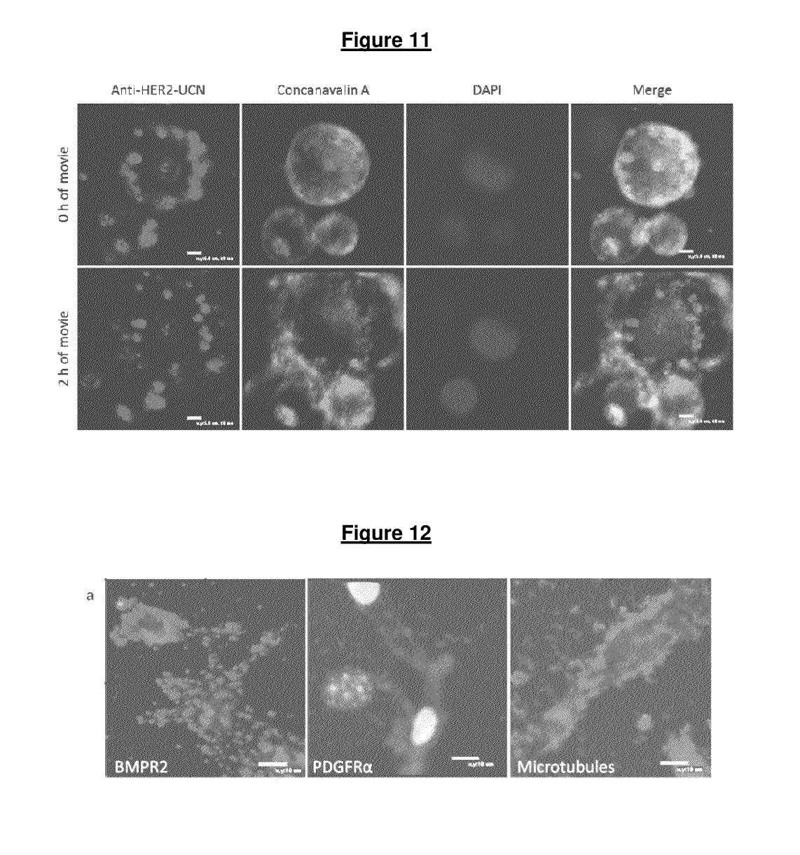

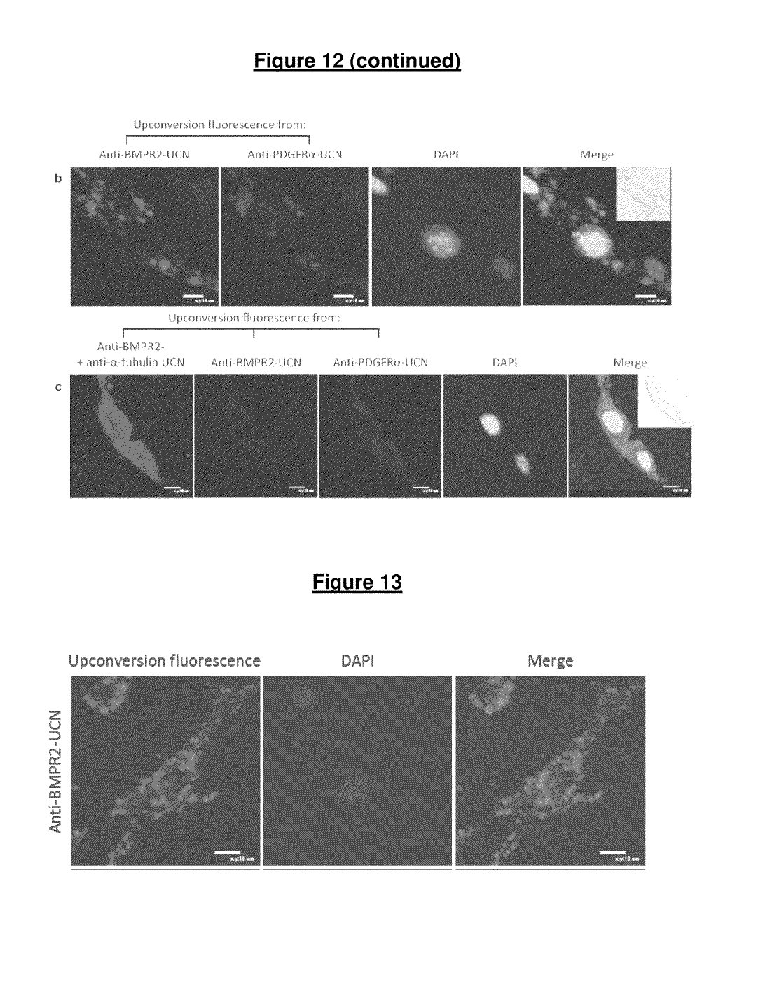

Having demonstrated that the UCNs are efficient labels in detecting single target with high specificity and photostability, its feasibility in multiplex detection of two cell surface receptors—BMPR2 and platelet derived growth factor receptor a (PDGFR α), and one intracellular structure—microtubule, simultaneously on a single sample of a 3T3 fibroblast cell line was established. To achieve this, UCNs having three different emission spectra (A:Yb,Tm@B:Er@A:Yb,Tm; A:Yb,Tm; and A:Yb,Er@B:Tm@A:Yb,Tm) were conjugated to the respective antibodies against BMPR2, PDGFR α and α-tubulin. Each of the three cellular targets was then singly labelled with these UCN-antibody conjugates before attempting on the multiplex detection set-up. As depicted in FIG. 12a and FIG. 13, fixed 3T3 cells incubated overnight with the UCNs displayed upconversion fluorescence with an overall output colour that is characteristic of each of their emission profile. It can be inferred here, that the UCN-antibody conjugates can be facilely adapted to applications on fixed specimens. Indeed, these observations are concordant with previous results and lend further support on its robustness to detect diverse array of cellular targets on a wide number of cells with different sample processing regimen.

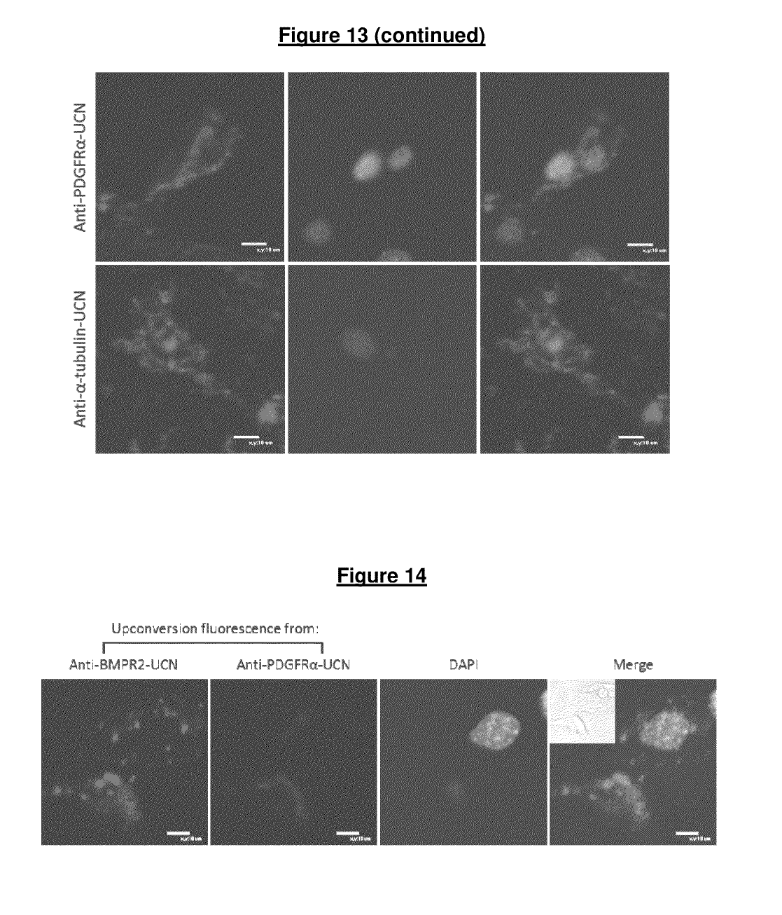

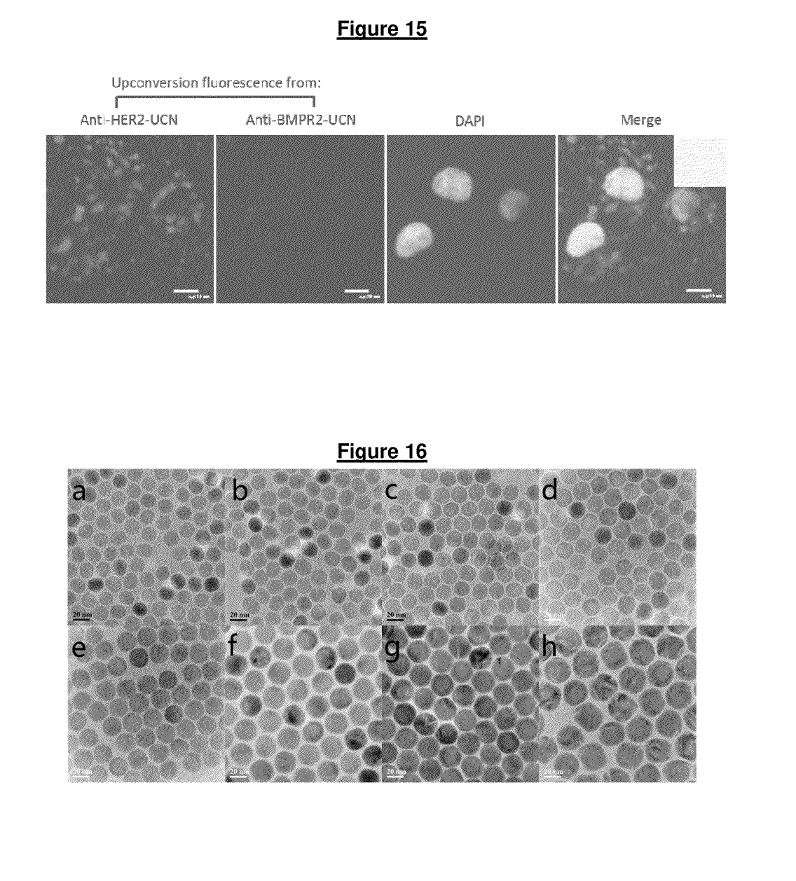

Next, two-colour multiplexing UCN system was set up to double-stain live 3T3 by incubating the cells with a cocktail of anti-BMPR2-UCNs-(A:Yb,Tm@B:Er@A:Yb,Tm) and anti-PDGFR α-UCNs-(A:Yb,Tm) for 4 hours. Under 980 nm excitation, two visually resolvable colours of the upconversion fluorescence were evident simultaneously in these cells (FIG. 12b and FIGS. 14 and 15), showing the spatially distinct distribution of BMPR2 and PDGFR α on 3T3 cells. Finally, the study was extended to a multiplexing system in which a third anti-α-tubulin-UCNs-(A:Yb,Er@B:Tm@A:Yb,Tm) was added alongside the previous anti-BMPR2- and anti-PDGFR α-UCNs to triple-stain stain fixed 3T3 cells. Again, upconversion fluorescence with a spectrally resolvable colour that is characteristic of each of the UCNs' emission profile was observed (FIG. 12c).

The amount of linkage between HER2 antibody covalently conjugated to carboxyl group functionalized upconversion fluorescent nanoparticles in which A:Yb,Tm@B:Er@A:Yb,Tm were also quantitatively confirmed by a modified Bradford assay (see below for more details) that showed 24.325 μg of HER2 antibody was conjugated to 0.005 mmol upconversion fluorescent nanoparticles as shown in FIG. 20.

The methods used for preparing the upconversion fluorescent nanoparticles may be as follows.

### A (NaYF₄) Core UCN Synthesis

All chemicals were purchased from Sigma-Aldrich (St. Louis, Mo., USA) and used without further purification. NaYF₄:20% Yb,2% Er (or 0.3% Tm) nanocrystals were synthesized following protocols reported previously with modification (Li et al, Advanced Materials, 2008; Qian and Zhang, Langmuir, 2008). 0.8 mmol YCl₃, 0.20 mmol YbCl₃ and 0.02 mmol ErCl₃ (or 0.003 mmol TmCl₃) were mixed with 6 ml oleic acid and 15 ml octadecene in a 50 ml flask. The solution was heated to 150° C. to form a homogeneous solution, and then cooled down to room temperature (RT). A solution of 4 mmol NH₄F and 2.5 mmol NaOH in 10 ml of methanol was next added into the flask and stirred for 30 minutes. Subsequently, the solution was slowly heated to remove the methanol followed by degassing at 100° C. for 10 minutes. It was then heated to 300° C. and maintained at that temperature for 1.5 hours under an argon atmosphere. The solution was allowed to cool to RT before the nanocrystals were precipitated out from the solution with acetone. They were then washed thrice with ethanol/water (1:1 v/v) and finally dispersed in cyclohexane for subsequent use.

### AB (NaYF₄@NaYbF₄) Core-Shell UCN Synthesis

1 mmol YbCl₃, and 0.02 mmol ErCl₃ (or 0.003 TmCl₃) were mixed with 6 ml oleic acid and 15 ml octadecene in a 50 ml flask. The solution was heated to 150° C. to form a homogeneous solution, and then allowed to cool down. Solution of the core nanocrystals dispersed in cyclohexane that was earlier obtained from the previous step was next added to the flask. The solution was maintained at 70° C. so as to remove the cyclohexane solvent and then subsequently cooled down to RT. Following this, a solution of 4 mmol NH₄F and 2.5 mmol NaOH in 10 ml of methanol was added into the flask and stirred for 30 minutes. Then, the solution was slowly heated to remove the methanol followed by degassing at 100° C. for 10 minutes. Subsequently, the solution was heated to 300° C. for 1.5 hours under an argon atmosphere. The solution was once again cooled down before the nanocrystals were precipitated out from the solution with acetone. This was washed thrice with ethanol/water (1:1 v/v) and the resultant AB nanocrystals were dispersed in cyclohexane for the next layer of coating.

### ABA (NaYF₄@NaYbF₄@NaYF₄)Sandwich Structured UCN Synthesis

0.8 mmol YCl₃, 0.20 mmol YbCl₃ and 0.02 mmol ErCl₃ (or 0.003 mmol TmCl₃) were mixed with 6 ml oleic acid and 15 ml octadecene in a 50 ml flask. The solution was heated to 150° C. to form a homogeneous solution, and then allowed to cool down. Core-shell AB nanocrystals dispersed in cyclohexane solution as obtained earlier from the previous step were next added into the flask. The solution was maintained at 70° C. to evaporate off the cyclohexane and then cooled down to RT. A solution of 4 mmol NH₄F and 2.5 mmol NaOH in 10 ml of methanol was added into the flask and stirred for 30 minutes. Subsequently, the solution was slowly heated to remove the methanol, degassed at 100° C. for 10 minutes, and then heated to 300° C. for 1.5 hours under an argon atmosphere. The solution was once again cooled down before the nanocrystals were precipitated out from the solution with acetone. This was washed thrice with ethanol/water (1:1 v/v). The resultant nanocrystals obtained are the sandwich structured ABA UCNs.

### Conjugation of Antibodies to Upconversion Fluorescent Nanoparticles (UCNs)

Antibodies were covalently conjugated to UCNs using the EDC-NHS chemistry. UCNs were first carboxylized with carboxyethyl silane triol sodium salt. 0.25 ml of CO-520, 4 ml of cyclohexane and 1 ml of 0.02 M ABA UCNs dispersed in cyclohexane were mixed in a bottle followed by sonication. 0.04 ml of ammonia (33 wt %) was then added into the bottle and this was sealed before it was shaken fiercely to form a transparent emulsion. 5 μl of TEOS and 5 μl of carboxyethyl silane triol sodium salt was next added into the solution followed by vigorous stirring of the solution at 60 rpm for two days. The product was precipitated out by ethanol, washed twice with ethanol/water (1:1 v/v) and then stored in water. 1 ml of 2 mM UCNs was activated with 1 μl of 0.2 mg/μl N-hydroxysuccinimide and 1 μl of 0.3 mg/μl 1-(3-dimethylaminopropyl)-3-ethylcarbodiimide hydrochloride with vigorous shaking for 15 minutes. After which, the activation buffer was removed and the mixture topped up with fresh DI water. 20 μl of 4 μg/μl antibody solution was added to the activated particles and incubated at 4° C. for 3 hours. Details of the antibodies are as follows: anti-HER2 (AbD Serotec, Kidlington, Oxford, UK); anti-BMPR2 (N-term) (Abgent, San Diego, Calif., USA); anti-PDGFR α (Cell Signaling Technology, Beverly, Mass., USA); anti-α-tubulin (Cell Signaling Technology, Beverly, Mass., USA). This was followed by washing of the particles twice with water, with centrifugation step at 5,000 rpm for 5 minutes in between the washings. Finally, the UCN-antibody conjugates were re-suspended and stored in 1 ml of DI water.

### Modified Bradford Assay

Standard solutions of HER2 antibody at four different concentrations of 0, 5, 10, 15 and 20 μg/ml were first prepared. These standards, together with a suspension of UCN-antibody conjugates (derived from above), were each mixed with Coomassie® Brilliant Blue G-250 dye (Bio-Rad) at a ratio of 4:1 with vortexing. After 5 minutes of incubation at room temperature, the samples were then each measured for their absorbance at 595 nm. The concentration of antibody conjugated to the UCNs was calculated based on the standard curve created from the absorption spectra of standard HER2 antibody solutions.

### Cell Culture

SK-BR-3 cells were grown in McCoy5A medium while MCF-7 and NIH-3T3 cells were grown in Dulbecco's Modified Eagle Medium at 37° C. in a humidified, 5% CO₂ atmosphere. All media were supplemented with 10% fetal bovine serum, 100 units/ml of penicillin and 100 μg/ml of streptomycin. One day before staining, the cells were seeded onto appropriate culture dishes at a plating density of 57,000 cells/cm² for SK-BR-3 and MCF-7 cells, and 30,000 cells/cm² for NIH-3T3 cells. Staining was done the next day as detailed below.

### Live Cell Staining

Previously seeded cells were incubated with either anti-HER2-UCNs-(A:Yb,Tm@B:Er@A:Yb,Tm), anti-BMPR2-UCNs-(A:Yb,Tm@B:Er@A:Yb,Tm), anti-PDGFR α-UCNs-(A:Yb,Tm) or a combination of these at a pre-optimized extracellular concentration of 0.342, 0.125 and 3.56 mM, respectively. Binding of UCNs onto the respective cell surface receptors was allowed to proceed by incubating them for 3 hours at 37° C. in a humidified, 5% CO₂ atmosphere. In the last 30 minutes of the incubation period, their plasma membrane was counterstained with 0.1 mg/ml Alexa Fluor 488-conjugated Concanavalin A and nuclei with 0.02 mg/ml DAPI. After which, the old culture medium containing unbound UCNs and excess counterstaining dyes were discarded. The cells were washed twice with culture medium and once with 1× phosphate buffered saline (PBS) before being fixed in 4% paraformaldehyde for 10 min at RT. This was followed by twice washing in 1×PBS for 5 minutes each.

### Fixed Cell Staining

Previously seeded cells were fixed in 4% paraformaldehyde for 10 minutes at RT. They were then rehydrated in 1×PBS for 5 min and this was repeated twice. For subsequent staining of cells with anti-α-tubulin-UCNs, the cells were subjected to an additional step of permeabilization in 0.1% Triton X-100 in PBS for 5 minutes at RT. Non-specific binding sites were blocked with 2% goat serum and 2% bovine serum albumin in 0.1% Tween 20 for 1 hour at 37° C. This was followed by incubation of the cells with either anti-BMPR2-UCNs-(A:Yb,Tm), anti-BMPR2-UCNs-(A:Yb,Tm@B:Er@A:Yb,Tm), anti-PDGFR α-UCNs-(A:Yb,Tm), anti-α-tubulin-UCNs-(A:Yb,Er@B:Tm@A:Yb,Tm) or a combination of these at a pre-optimized extracellular concentration of 3.56, 0.125, 3.56 and 1.78 mM, respectively. Binding of UCNs onto the respective cellular markers was allowed to proceed by incubating them overnight at 4° C. The following day, cells were washed thrice with 1×PBS. Their nuclei were counterstained with 0.1 μg/ml DAPI for 5 minutes at RT followed by twice washing with 1×PBS for 5 minutes each.

### Confocal Imaging of UCN-Stained Cells

UCNs, DAPI and Alexa Fluor 488 stainings on the cells were visualized by excitation at 980, 408 and 488 nm, respectively using a confocal laser scanning microscope (Nikon C1 Confocal, Nikon Inc., Tokyo, Japan) specially fitted with a continuous wave 980 nm laser excitation source (Opto-Link Corp., Hong Kong).

The upconversion fluorescent nanoparticle according to the present disclosure may be suitable for several applications. For example, the upconversion fluorescent nanoparticles may be suitable for, but not limited to, photoactivable gene therapy, photochemical internalization, photo-activated ion channels, photodynamic therapy, etc. Multicolour upconversion nanoparticles can be used as versatile fluorescent labels for multiplex bio-imaging and bio-assay applications, for example, to develop kits for multiplex detection and quantitative measurement of biomarkers. Other applications of these nanoparticles include, but are not limited to, for example, computing and memory; electronics and displays; optoelectronic devices such as LEDs, lighting, and lasers; optical components used in telecommunications; and security applications such as covert identification tagging or biowarfare detection sensors.

According to a second aspect, the present disclosure provides an article of manufacture comprising the upconversion fluorescent nanoparticles described above. The article of manufacture may be any suitable article. For example, the article of manufacture may be, but not limited to, a bio-probe, a carrier for drug delivery, a device for bio-imaging, a bioassay, a device for bio-detection, or an optoelectronic device.

According to a third aspect, the present disclosure provides a bio-imaging and/or bio-detection apparatus comprising at least one upconversion fluorescent nanoparticle described above; at least one biomolecule; and at least one source of excitation. The biomolecule may be any suitable biomolecule. For example, the biomolecule may be as described above. The at least one source of excitation may be any suitable source. For example, the source of excitation may be NIR. In particular, the NIR may be at 980 nm.

Whilst the foregoing description has described exemplary embodiments, it will be understood by those skilled in the technology concerned that many variations in details of design, construction and/or operation may be made without departing from the present disclosure.

### REFERENCES

1. Li Z Q et al., Multicolor core/shell-structured upconversion fluorescent nanoparticles, Advanced Materials, 2008, 20:4765-4769;
2. Qian H S and Zhang Y, Synthesis of hexagonal-phase core-shell NaYF₄ nanocrystals with tunable upconversion fluorescence, Langmuir, 2008, 24:12123-12125.

1. An upconversion fluorescent nanoparticle comprising: a first nanocrystal layer, a second nanocrystal layer, and an energy absorbing layer disposed between the first nanocrystal layer and the second nanocrystal layer, wherein each of the first nanocrystal layer and the second nanocrystal layer comprises at least one compound of formula (M₁)ⱼ(M₂)ₖXₙ:(M₃)_{q}, and the energy absorbing layer comprises at least one compound of formula (M₁)ⱼ(M₂)ₖXₙ:(M₃)ᵣ, wherein
each X is the same or different and is selected from the group consisting of: halogen, O, S, Se, Te, N, P and As;
each M₁, if present is the same or different and is selected from the group consisting of: Li, Na, K, Rb, Cs, Fr, Be, Mg, Ca, Sr, Ba, Ra, O and NH₄;
each M₂ is the same or different and is a metal ion;
each M₃, independently, is the same or different and is selected from the group consisting of Er, Tm, Pr, Ho, Nd, Tb, Eu, Sm, Yb, Ce, Dy, Mo, and Cs;
j is 0≦j≦10; k is 1≦k≦10; n is 1≦n≦10; q is 1≦q≦10; and r is 0≦r≦10.

2. The upconversion fluorescent nanoparticle according to claim 1, wherein M₂ is selected from the group consisting of: transition metal ions, inner transition metal ions, and Group I to VI metal ions.

3. The upconversion fluorescent nanoparticle according to claim 1, wherein each of the first nanocrystal layer, the second nanocrystal layer, and the energy absorbing layer comprises at least one emitter ion and at least one absorber ion.

4. The upconversion fluorescent nanoparticle according to claim 1, wherein the energy absorbing layer is saturated with at least one absorber ion.

5. The upconversion fluorescent nanoparticle according to claim 1, wherein the first nanocrystal layer and the second nanocrystal layer is selected from the group consisting of: NaYF₄:(M₃)_{q}, La₂O₃:(M₃)_{q}, La₂O₃:(M₃)_{q}, La₂(MoO₄)₃:(M₃)_{q}, LnF₃:(M₃)_{q}, Y₂O₂S:(M₃)_{q}, Y₂O₃:(M₃)_{q}, TeO₂:(M₃)_{q}, ZrO₂:(M₃)_{q}, LaPO₄:(M₃)_{q}, and LiYF₄:(M₃)_{q}, wherein M₃ and q are as defined in claim 1.

6. The upconversion fluorescent nanoparticle according to claim 1, wherein the energy absorbing layer is selected from the group consisting of: NaYbF₄:(M₃)ᵣ, La₂O₃:(M₃)ᵣ, La₂O₃:(M₃)ᵣ, La₂(MoO₄)₃:(M₃)ᵣ, LnF₃:(M₃)ᵣ, Y₂O₂S:(M₃)ᵣ, Y₂O₃:(M₃)₁, TeO₂:(M₃)ᵣ, ZrO₂:(M₃), LaPO₄:(M₃)ᵣ, and LiYbF₄:(M₃)ᵣ, wherein M₃ and r are as defined in claim 1.

7. The upconversion fluorescent nanoparticle according to claim 1, wherein each of the first nanocrystal layer and the second nanocrystal layer is the same or different and is selected from the group consisting of: NaYF₄:Yb,Er and NaYF₄:Yb,Tm, and the energy absorbing layer is selected from the group consisting of: NaYbF₄, NaYbF₄:Er, NaYbF₄:Tm and NaYbF₄:Ho.

8. The upconversion fluorescent nanoparticle according to claim 1, further comprising at least one biomolecule attached to the nanoparticle.

9. The upconversion fluorescent nanoparticle according to claim 8, wherein the biomolecule is selected from the group consisting of: protein, nucleic acid, nucleosides, nucleotides, DNA, hormone, amino acid, peptide, peptidomimetic, RNA, lipid, albumin, antibody, phospholipids, glycolipid, sterol, vitamins, neurotransmitter, carbohydrate, sugar, disaccharide, monosaccharide, oligopeptide, polypeptide, oligosaccharide, polysaccharide, and a mixture thereof.

10. The upconversion fluorescent nanoparticle according to claim 1, wherein the nanoparticle is a NIR-to-visible, NIR-to-NIR, or NIR-to-ultraviolet upconversion fluorescent nanoparticle.

11. An article of manufacture comprising the upconversion fluorescent nanoparticle according to claim 1.

12. The article of manufacture according to claim 11, wherein the article of manufacture is a bio-probe, a carrier for drug delivery, a device for bio-imaging, a bioassay, a device for bio-detection, or an optoelectronic device.

13. A bio-imaging and/or bio-detection apparatus comprising at least one upconversion fluorescent nanoparticle according to claim 1; at least one biomolecule; and at least one source of excitation.
